(19) United States
(12) Patent Application Publication
(10) Pub. No.: US 2021/0386787 A1
(43) Pub. Date: Dec. 16, 2021

(54) Hematopoietic Stem and Progenitor Cell Expansion System

(71) Applicant: LIFE TECHNOLOGIES CORPORATION, Carlsbad, CA (US)

(72) Inventors: Janet SEI, Clarksburg, MD (US); Abigail BECKER, Fairfield, PA (US); Navjot KAUR, Frederick, MD (US); Mohan VEMURI, Frederick, MD (US)

(21) Appl. No.: 17/226,642

(22) PCT Filed: Oct. 14, 2019
(86) PCT No.: PCT/US2019/056145

§ 371 Date: Apr. 9, 2021

Related U.S. Application Data
(60) Provisional application No. 62/745,208, filed on Oct. 12, 2018.
(60) Provisional application No. 62/854,250, filed on May 29, 2019.

(51) Int. Cl. A61K 35/28; C12N 5/0789; C12N 5/00

(52) U.S. Cl. CPC A61K 35/28; C12N 5/0647; C12N 5/0037; C12N 2501/065; C12N 2501/125; C12N 2510/00; C12N 2501/2303; C12N 2501/2306; C12N 2501/26; C12N 2501/72; C12N 2501/145

(57) ABSTRACT

Described herein is a growth medium for culture of stem cells and/or primary cells, in particular hematopoietic stem cells (HSCs), the growth medium including a basal medium and a supplement, with the medium and/or supplement including a histone acetyltransferase (HAT) inhibitor, a histone deacetylase (HDAC) inhibitor, and two or more of a lipid, an amino acid or amino acid derivative, an antioxidative agent, and an inorganic salt. Further provided are methods of using the growth medium, as well as kits and formulations of the growth medium.

### CROSS-REFERENCE TO RELATED APPLICATIONS

This application is a U.S. 371 National Phase Application and claims priority to PCT/US2019/056145 filed Oct. 14, 2019, which claims priority to and the benefit of U.S. Provisional Application No. 62/854,250 filed May 29, 2019, and U.S. Provisional Application No. 62/745,208 filed Oct. 12, 2018. The contents of each of the aforementioned applications are incorporated herein by reference in their entirety.

### BACKGROUND

Cell culture media provide the nutrients necessary to maintain and grow cells in a controlled, artificial and in vitro environment. Characteristics and formulations of cell culture media vary depending upon the particular cellular requirements. Important parameters include osmolality, pH, and nutrient compositions.

Cell culture medium formulations have been well documented in the literature and a large number of media are commercially available. Typical components of cell culture media include amino acids, organic and inorganic salts, vitamins, trace metals, sugars, lipids and nucleic acids, the types and amounts of which may vary depending upon the particular requirements of a given species, cell or tissue type.

Multipotent human hematopoietic stem/progenitor cells (HSPCs, also called HSCs) are required for various applications of experimental transplantation, graft studies and to differentiate them further into all blood lineages, including myeloid (red blood cells, macrophages, platelets) and lymphoid cells (e.g., T cells and B cells). CD34 is a marker of HSCs that is routinely used to identify and isolate HSCs. A major limitation to the usage of primary HSCs is the low CD34⁺ cell numbers present in tissues, such as cord blood and peripheral blood, that are generally used for isolation of HSCs. See, for example, Bhattacharya et al. (2008) European Journal of Immunology 38(8): 2060-2067; Notta et al. (2011) Science 333(6039):218-221; Doulatov et al. (2012) Cell Stem Cell 10(2):120-136; each of which is incorporated herein by reference in its entirety.

There remains a need for chemically-defined cell medium useful for expanding HSCs and other primary cells, in particular to obtain a population of HSCs with a high proportion of Lineage⁻ CD34⁺CD90⁺CD45RA⁻CD49f⁺ (CD34⁺CD90⁺CD45RA⁻) cells that are considered long term repopulating HSCs.

### SUMMARY

Currently, there are media systems that expand the number of CD34⁺ cells ex vivo. However, with commercially available media systems, the expansion of hematopoietic stem cells (HSCs) occurs disproportionately in that the majority of the resultant cells are short term HSC, with limited long term HSCs present. Some embodiments described herein relate to a media system that can effectively expand both long term and short term human HSCs ex vivo. In addition, some embodiments of the present disclosure can enable the expansion of human HSCs from different tissue sources such as cord blood, mobilized peripheral blood, and bone marrow. Formulation embodiments are fully developed and HSCs derived or expanded with the various embodied media may be used for one or more of biochemical, transcriptomic, epigenetic, and transplantation studies, for example.

In an aspect, a growth medium is provided for culture of hematopoietic stem cells (HSCs), including a basal medium and a supplement, with the medium and/or supplement including a histone acetyltransferase (HAT) inhibitor, a histone deacetylase (HDAC) inhibitor, and two or more of a lipid, an amino acid or amino acid derivative, an antioxidative agent, and an inorganic salt.

In embodiments, the basal medium is selected from OPTMIZER™ CTS™ T-Cell Expansion serum-free medium, Dulbecco's Modified Eagle Media (DMEM), Iscove's Modified Dulbecco's Medium (IMDM), DMEM/F12, Advanced DMEM/F12, and KNOCKOUT™ DMEM/F12.

In embodiments, the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin. In certain embodiments, the HAT inhibitor is present at a concentration of between 0.001 grams/liter (g/L) and 1 g/L. In certain embodiments, each of the previously mentioned HAT inhibitors may independently be excluded from a medium and/or supplement as described herein.

In embodiments, the HDAC inhibitor is selected from apicidin, belinostat, CI-994, CRA-024781, panobinostat, sodium butyrate, sodium phenylbutyrate, suberoylanilide hydroxamic acid (vorinostat), trichostatin A, sodium valproate (valproic acid; VPA), givinostat, MS-275, MGCD0103, and Scriptaid. In certain embodiments, the HDAC inhibitor is present at a concentration between 0.0005 g/L and 10 g/L. In certain embodiments, each of the previously mentioned HDAC inhibitors may independently be excluded from a medium and/or supplement as described herein.

In embodiments, the HDAC inhibitor and HAT inhibitor are at a weight ratio of 1:1 to 1:30 HDAC inhibitor:HAT inhibitor.

In embodiments the inorganic salt is selected from a copper salt, a magnesium salt, a selenite salt, a potassium salt, a calcium salt, a zinc salt, an iron salt, a sodium salt, or combinations thereof. In certain embodiments, each of the previously mentioned inorganic salts may independently be excluded from a medium and/or supplement as described herein.

In embodiments, the lipid is selected from cholesterol, linoleic acid, linolenic acid, oleic acid, palmitic acid, arachidonic acid, palmitoleic acid, myristic acid, and combinations thereof. In certain embodiments, each of the previously mentioned lipids may independently be excluded from a medium and/or supplement as described herein.

In embodiments, the growth medium also includes albumin, insulin, transferrin, interleukin 3 (IL-3), interleukin 6 (IL-6), stem cell factor, Fms-related tyrosine kinase 3 ligand, thrombopoietin, granulocyte colony-stimulating factor, granulocyte-macrophage colony-stimulating factor, and/or combinations thereof. In certain embodiments, each of the previously mentioned additional ingredients may independently be excluded from a medium and/or supplement as described herein.

In embodiments, the antioxidative agent is selected from polyphenols, ascorbate, and carotenoids. In embodiments, the polyphenol is selected from those found naturally in fruits, wines, and teas. In embodiments, the ascorbate is selected from ascorbate or ascorbic acid. In embodiments, the carotenoid is selected from beta-carotene, alpha-carotene and lycopene. In embodiments, the antioxidative agent is selected from DL Lipoic Acid, DL Tocopherol Acetate, and Ascorbic Acid.

In embodiments, the medium does not include an ingredient derived from an animal. In embodiments, the medium does not include serum. In embodiments, the medium is a xeno-free medium.

In an aspect, a kit is provided, the kit including a basal medium and a supplement, the medium and/or supplement including a histone acetyltransferase (HAT) inhibitor, a histone deacetylase (HDAC) inhibitor, and two or more of a lipid, an amino acid or amino acid derivative, an antioxidative agent, and an inorganic salt. In embodiments, the supplement includes a HAT inhibitor. In embodiments, the supplement includes a HDAC inhibitor. In embodiments, the basal medium includes a HAT inhibitor. In embodiments, the basal medium includes a HDAC inhibitor.

In an aspect, a kit is provided, the kit including a histone acetyltransferase (HAT) inhibitor, a histone deacetylase (HDAC) inhibitor, a basal medium, and a supplement, the medium and/or supplement including two or more of a lipid, an amino acid or amino acid derivative, an antioxidative agent, and an inorganic salt. In embodiments, each component of the kit (HAT inhibitor, HDAC inhibitor, supplement, and basal medium) is present in the kit in a separate container. In embodiments, any two or more of the components are present in the kit in the same container.

In embodiments, the supplement in the kit is at a concentration greater than 1×. As used herein, the term “lx” refers to a composition that is at a working concentration. That is, the composition is at the concentration recommended for use ((e.g., growing HSCs). In embodiments, the supplement in the kit is at a 2-fold to 100-fold concentration.

In an aspect, a growth medium supplement is provided for the culture of hematopoietic stem cells (HSCs), the growth medium supplement including a histone acetyltransferase (HAT) inhibitor and a histone deacetylase (HDAC) inhibitor. In embodiments, the growth medium supplement further includes a lipid, an amino acid or amino acid derivative, an antioxidative agent, and an inorganic salt. In embodiments, the growth medium supplement includes a HDAC inhibitor and a HAT inhibitor at a weight ratio of 1:1 to 1:30 HDAC inhibitor:HAT inhibitor.

In an aspect, a method of expanding stem cells is provided, the method including growing stem cells in a growth medium as described herein.

In embodiments, the stem cells are hematopoietic stem cells (HSCs). In embodiments, CD34-expressing HSCs are expanded between 20-fold and 350-fold after 4 to 14 days in culture. In embodiments, CD34-expressing HSCs are expanded between 50-fold and 350-fold after 4 to 14 days in culture. In embodiments, CD34-expressing HSCs are expanded between 80-fold and 350-fold after 4 to 14 days in culture. In embodiments, CD34-expressing HSCs are expanded between 100-fold and 350-fold after 4 to 14 days in culture. In embodiments, CD34-expressing HSCs are at least 50% of total nucleated cells after 7 days in culture. In embodiments, CD34-expressing HSCs are between 60% and 90% of total nucleated cells after 7 days in culture. In embodiments, CD34⁺CD90⁺CD45RA⁻ HSCs are enriched to at least 5% of total nucleated cells after 7 days in culture. In embodiments, CD34⁺CD90⁺CD45RA⁻ HSCs are enriched to at least 10% of total nucleated cells after 7 days in culture.

In an aspect, a method of expanding stem cells is provided, the method including (i) adding a supplement including a histone acetyltransferase (HAT) inhibitor and a histone deacetylase (HDAC) inhibitor to a basal medium to form a growth medium, and (ii) growing stem cells in the growth medium, thereby expanding the stem cells. In embodiments, the growth medium further comprises two or more of a lipid, an amino acid or amino acid derivative, an antioxidative agent, and an inorganic salt. In embodiments, the growth medium comprises a lipid, an amino acid or amino acid derivative, an antioxidative agent, and an inorganic salt.

In an aspect, a method of expanding primary cells from a subject is provided, the method including growing the primary cells in a growth medium including a basal medium, a histone acetyltransferase (HAT) inhibitor and a histone deacetylase (HDAC) inhibitor, and two or more of a lipid, an amino acid or amino acid derivative, an antioxidative agent, and an inorganic salt. In embodiments, the growth medium comprises a lipid, an amino acid or amino acid derivative, an antioxidative agent, and an inorganic salt. In embodiments, the primary cells are HSCs. In embodiments, the primary cells are immune cells. In embodiments, the immune cells are T cells, B cells, macrophages, NK cells, dendritic cells, monocytes, and/or granulocytes. In embodiments, the expanded primary cells are used in a method to treat a patient in need thereof, comprising administering the expanded primary cells. In embodiments, the primary cells are genetically modified before or after expansion.

In an aspect, a method of treating a subject in need of a therapy is provided, the method including (a) obtaining hematopoietic stem cells (HSCs); (b) expanding the HSCs in a growth medium as described herein comprising a HAT inhibitor and a HDAC inhibitor; and (c) transferring the HSCs to the subject, thereby treating the subject. In embodiments, the HSCs are derived from the subject. In embodiments, the method further includes genetically modifying the HSCs prior to transferring the HSCs to the subject. In embodiments, the subject has a hematopoietic malignancy and/or has undergone chemotherapy. In embodiments, the subject is human. In embodiments, the therapy is treatment of a cancer, an autoimmune disease, or a blood-based disease.

In an aspect, a method for editing a genome in a stem cell (SC) is provided, the method including: (a) obtaining a SC that was expanded in a growth media as described herein comprising a HAT inhibitor and a HDAC inhibitor; and (b) editing the genome of the stem cell. In embodiments, the genome is edited using one or more genome editing reagents selected from a zinc-finger nuclease (ZFN), transcription activator-like effector nuclease (TALEN), meganuclease, and a clustered regularly interspaced short palindromic repeat (CRISPR) associated protein.

In an aspect, HSCs expanded according to a method provided herein are used for the preparation of a medicament to treat a subject. In some embodiments, HSCs expanded using a growth medium comprising a basal medium, a histone acetyltransferase (HAT) inhibitor, and a histone deacetylase (HDAC) inhibitor, and at least two of a lipid, an amino acid or amino acid derivative, an antioxidative agent, and an inorganic salt are used for the preparation of a medicament to treat a subject in need thereof. In some embodiments, HSCs were derived from the subject to be treated prior to expansion in vitro.

In an aspect, a method for improving engraftment potential of a population of hematopoietic stem cells (HSCs) is provided, the method including: (a) obtaining a population of HSCs; and (b) expanding the population of HSCs using in a growth media as described herein comprising a HAT inhibitor and a HDAC inhibitor, thereby improving engraftment potential of the population of HSCs.

In an aspect, a method of treating a subject in need of a therapy is provided, the method comprising: (a) obtaining hematopoietic stem cells (HSCs) that were expanded using in a growth media as described herein comprising a HAT inhibitor and a HDAC inhibitor; and (b) transferring the HSCs to the subject, thereby treating the subject.

### BRIEF DESCRIPTION OF THE DRAWINGS

FIG. 1. Process Workflow for Expansion of CD34⁺ cells. CD34⁺ cells were expanded with HSC expansion media and characterized to determine the total nucleated cells (TNC), % Viability, phenotype and function.

FIGS. 2A-2F. Analysis of Existing solutions in Expansion of Umbilical Cord Blood CD34⁺ cells. Representative results of current solutions demonstrated that the highest increase in HSC was observable at 7 days post-expansion. Viability of cells is highest at day 7 (FIG. 2A). While the number of total nucleated cells (TNC) increases with time (FIG. 2B), the percentage of CD34⁺ in the population of expanded cells decreases due to differentiation of HSC (FIG. 2C). However, the fold expansion of CD34⁺ was highest at Day 7 post-expansion (FIG. 2D). Although the highest frequency of CD34⁺CD90⁺CD45RA⁻ cells was observed at Day 5 (as a percentage of TNC; FIG. 2E), the highest fold increase in the number of CD34⁺CD90⁺CD45RA⁻ cells was observed at Day 7 (FIG. 2F).

FIG. 3 shows the increase in CD34⁺ HSCs (as fold CD34⁺) when cells are expanded in media±Garcinol and/or VPA, in HSC Expansion Medium compared to the indicated growth media.

FIG. 4 shows FACS graphs for HSCs expanded in basal media±Garcinol and/or VPA using HSC Expansion Medium or the indicated growth media.

FIG. 5 shows a bar graph of the percentage of TNC that are CD34⁺CD90⁺CD45RA⁻ after expansion in basal media±Garcinol and/or VPA, based on the FACS analysis of FIG. 4.

FIGS. 6A-6B. FIG. 6A shows fold increase in TNC when HSC are expanded in basal media±Garcinol and/or VPA, using HSC Expansion Medium compared to the indicated growth media. FIG. 6B shows the percentage of total nucleated cells that are CD34⁺ when grown in basal media±Garcinol and/or VPA using HSC Expansion Medium compared to the indicated growth media.

FIGS. 7A-7E. Competitive comparison of phenotypic performance data. Expansion of HSCs from human mobilized peripheral blood (mPB) in HSC Medium provides approximately 100-fold increase in CD34⁺ cells. Expansion of CD34⁺ mPB in HSC Medium led to significantly higher levels of expansion of CD34⁺ cells (FIG. 7A) and TNC (FIG. 7B), with cells demonstrating >80% viability (FIG. 7C). Expanded cells were >60% CD34⁺ (FIG. 7D), with significantly higher levels of CD34⁺CD90⁺CD45RA⁻ long term HSC (FIG. 7E). Percentages of CD34⁺ and CD34⁺CD90⁺CD45RA⁻ shown are from the total live TNC population. Three human single donor purified CD34⁺ mPB were cultured in HSC Medium (HSC Basal and 50× Supplement) or three commercial media, all supplemented with growth factors (SCF, Flt3L, TPO, IL-3, and IL-6). Cells were cultured for 7 days, following which total nucleated cells (TNC) and % viability were determined using a Countess II, and phenotype assessed as described. Error bars denote standard deviation.

FIGS. 8A-8E. Lot to lot phenotypic performance data. HSC Medium demonstrates Consistent Lot-to-Lot Performance. CD34⁺ mPB expanded in three different lots of HSC Medium demonstrate equivalent levels of CD34⁺ cell expansion (FIG. 8A), TNC expansion (FIG. 8B), % viability (FIG. 8C), % CD34⁺ (FIG. 8D), and % CD34⁺CD90⁺CD45RA⁻ long term HSC (FIG. 8E). Three human single donor purified CD34⁺ mPB were cultured in three different lots of HSC Medium (HSC Basal and 50× Supplement), all supplemented with growth factors. Cells were cultured for 7 days, following which total nucleated cells (TNC) and % viability were determined using a Countess II, and phenotype assessed as described in FIG. 9. Error bars denote standard deviation.

FIG. 9. Phenotypic analysis gating strategy. Phenotypic Characterization of Expanded HSC Cultured in HSC Medium. Purified CD34⁺ from mPB were cultured in HSC Medium containing growth factors. Cells were cultured for 7 days, then assessed by flow cytometry for expression of CD34, CD90, and CD45RA. Doublets and dead cells were excluded from analysis, and gates identifying CD34⁺ cells and CD90⁺CD45RA⁻ cells were demarcated based on fluorescent minus one (FMO) controls.

FIG. 10. Colony Forming Unit (CFU) assay data. CD34⁺ cells expanded in HSC Medium maintain in vitro differentiation capacity. CD34⁺ mPB expanded in HSC Medium were able to differentiate into Granulocyte/Erythroid/Monocyte/Megakaryocyte (GEMM), Erythroid (E), and Granulocyte/Monocyte (GM) colony forming cells during a 14 day culture period. Two human single donor purified CD34⁺ mPB were cultured for 7 days in HSC Medium, supplemented with growth factors. Subsequently, expanded TNC were cultured for another 14 days in semi-solid medium to assess for Colony forming cells (CFC). Images show examples of colonies identified.

FIGS. 11A-11E. HSC Medium Expands Single donor human CD34⁺ mPB cells. All three human single donor CD34⁺ mPB that were expanded in HSC Medium demonstrated equivalent levels of expansion in CD34⁺ cells (FIG. 11A), TNC (FIG. 11B), % viability (FIG. 11C), % CD34⁺ (FIG. 11D). Notably, the level of expansion of CD34⁺CD90⁺CD45RA⁻ long term HSC (FIG. 11E) varied amongst donors. Three human single donor purified CD34⁺ mPB were cultured in HSC Medium, supplemented with growth factors. Cells were cultured for 7 days, following which total nucleated cells (TNC) and % viability were determined using a Countess II, and phenotype assessed as described in FIG. 9. Error bars denote standard deviation within sample replicates.

FIGS. 12A-12D. CD34⁺ cells Expanded in HSC Medium express highest ALDH levels. CD34⁺ mPB expanded in HSC Medium were assessed for expression of Aldehyde dehydrogenase (ALDH). Gating shown to demonstrate identification of cells incubated in (FIG. 12A) control DEAB with no ALDH expression, compared to (FIG. 12B) ALDEFLUOR™ positive cell populations. Expansion of CD34⁺ in HSC Medium demonstrated (FIG. 12C) highest ALDH expression on a per-cell basis (Geometric Mean Fluorescent Intensity), and (FIG. 12D) highest % of CD34⁺ cells staining positive for ALDH. Consistency in ALDH expression by expanded cells was observed between HSC Medium lots. Three human single donor purified CD34⁺ mPB were cultured in three lots of HSC Medium or three commercial media, all supplemented with growth factors. Cells were cultured for 7 days, following which total nucleated cells (TNC) were incubated in either DEAB or ALDEFLUOR™, according to manufacturer's instructions. Cells were stained with antibodies to identify CD34⁺ cells, and analyzed for ALDH expression. Data displayed were pooled from the three single donor mPB cells. Error bars denote standard error.

FIGS. 13A-D. CD34+ cells expanded in HSC Medium can be genetically engineered. FIG. 13A is a bar graph showing the transfection efficiency of CD34+ cells from mobilized peripheral blood (mPB) from three single donors (P2, P8, and P23). CD34+ cells expanded in HSC media were electroporated with Cas9, guide RNA, and a GFP donor DNA (100 ng/ml, 200 ng/ml, or 500 ng/ml) as indicated, as described in Example 4. The % GFP⁺ cells of each sample was calculated (FIG. 13A). Error bars denote standard error. Transfected cells were sorted (GFP+ or GFP−). Sorted cells were stained with antibodies to identify CD34+CD90+ cells. The percentage of CD34+CD90+ cells successfully modified by Cas9 (GFP+) was similar to the percentage of CD34+CD90+ cells that had not been genetically modified (GFP−)(FIG. 13B), demonstrating that genetically modification does not impact the phenotype of these HSC populations. Moreover, these data demonstrate successful CRISPR-mediated modification of CD34+CD90+ cells and CD34+CD90− cells. GFP+ and GFP− cells were subjected to an in vitro colony forming assay as described in Example 4. FIG. 13C is a bar graph showing the number of erythroid (red) and granulocytic/monocytic (white) colony forming units/1000 cells after 14 day culture in METHOCULT™ media (Stem Cell Technologies, Inc). The number of colony forming cells/1000 cells was similar for the two groups analyzed (unsorted, GFP+ sorted). FIG. 13D are photographs of colonies identified from the GFP+ population using a fluorescent microscope and bright-field microscope.

FIGS. 14A and 14B. STEMPRO™ HSC Media Enables Reprogramming of CD34+ Cells into iPSC with CTS CytoTune 2.1. FIG. 14A is a bar graph showing the percent reprogramming efficiency of CD34+ cells expanded in OPTMIZER™ or STEMPRO™ HSC culture media as described in Example 5 herein. FIG. 14B are photographs of reprogrammed iPSC colonies stained with alkaline phosphatase and imaged to view the number of colonies cultured in OPTMIZER™ or STEMPRO™ HSC Expansion media as described in Example 5 herein. The reprogramming efficiency of CD34+ cells expanded in HSC expansion media as described herein was superior (approximately 10-fold) to the reprogramming efficiency of CD34+ cells expanded in OPTMIZER™ culture media.

FIG. 15. STEMPRO™ HSC Expansion Media Enables Reprogramming of PBMC into iPSC with CTS™ CytoTune 2.1 & CytoTune 2.0 iPSC reprogramming kits. FIG. 15 is a bar graph showing the percent reprogramming efficiency of PBMCs expanded in STEMPRO34™ or STEMPRO™ HSC Expansion Medium as described in Example 6 herein. PBMCs expanded in STEMPRO™ HSC Expansion Medium (as described herein) exhibited superior reprogramming efficiency compared to PBMCs expanded in STEMPRO-34 culture medium.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### I. Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terms “a” or “an,” as used in herein means one or more.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Optional” or “optionally” means that the subsequently described event or circumstance can or cannot occur, and that the description includes instances where the event or circumstance occurs and instances where it does not.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulinoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “inflammatory disease” refers to a disease or condition characterized by aberrant inflammation (e.g. an increased level of inflammation compared to a control such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, asthma, allergic asthma, acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

A “stem cell” is a cell characterized by the ability of self-renewal through mitotic cell division and the potential to differentiate into a tissue or an organ. Among mammalian stem cells, embryonic stem cells (ES cells) and somatic stem cells (e.g., HSC) can be distinguished. Embryonic stem cells reside in the blastocyst and give rise to embryonic tissues, whereas somatic stem cells reside in adult tissues for the purpose of tissue regeneration and repair.

In embodiments, HSCs can be used to treat diseases including, but not limited to: aplastic anemia, Fanconi anemia, Diamond-blackfan syndrome, Sickle cell disease, Thalassemia, Paroxysmal nocturnal hemoglobinuria, Chediak-Higashi syndrome, Chronic granulomatous disease, Glanzmann thrombasthenia, Osteopetrosis, Lysosomal storage disorders, Gaucher disease, Niemann-Pick, Mucopolysaccharidosis, Glycoproteinoses, Immune deficiencies, Ataxia telangiectasia, DiGeorge syndrome, Severe combined immunodeficiency (SCID), Wiscott-Aldrich, Kostmann syndrome, Shwachman-Diamond syndrome, Leukemias, Acute myelogenous leukemia, Acute lymphoblastic leukemia, Hairy cell leukemia, Chronic lymphocytic leukemia, Myelodysplasia, Lymphomas, Hodgkin disease, Non-Hodgkin lymphoma, Multiple myeloma, Myeloproliferative neoplasms, Myelofibrosis, Polycythemia vera, Myelofibrosis, Chronic myelogenous leukemia, Solid tumors, Neuroblastoma, Desmoplastic small round cell tumor, Ewing sarcoma, and Choriocarcinoma.

The term “serum-free” as used herein refers to medium which is free or substantially free of serum. “Substantially free of serum” as used herein refers to media which contains less than about 1% serum by weight, contains only trace amounts of serum, or contains undetectable amounts of serum.

The term “chemically-defined medium” as used herein refers to medium suitable for in vitro culture of cells, particularly eukaryotic cells, in which all of the chemical components and their concentrations are known.

The phrase “protein-free” culture medium refers to culture medium that contain no protein (e.g., no serum proteins such as serum albumin or attachment factors, nutritive proteins such as growth factors, or metal ion carrier proteins such as transferrin, ceruloplasmin, etc.). Preferably, if peptides are present, the peptides are smaller peptides, e.g., di- or tri-peptides. Preferably, peptides of deca-peptide length or greater are less than about 1%, more preferably less than about 0.1%, and even more preferably less than about 0.01% of the amino acids present in the protein free medium.

The term “animal derived” material as used herein refers to material that is derived in whole or in part from an animal source, including recombinant animal DNA or recombinant animal protein DNA.

By “cell culture” or “culture” is meant the maintenance of cells in an artificial, in vitro environment.

By “cultivation” is meant the maintenance of cells in vitro under conditions favoring growth and/or differentiation and/or or continued viability. “Cultivation” can be used interchangeably with “cell culture.” Cultivation is assessed by number of viable cells/mL culture medium.

By “expansion” is meant the growth of cells in culture to increase the number of the cells from an initial cell number to a larger cell number after culture. The cell number may include, without limitation, total cells, total viable cells, total nucleated cells, or any combination thereof.

The term “replenishing, replacing, or supplementing medium” as used herein refers to adding a volume of fresh cell culture medium to medium that was already present in culture and/or replacing medium that was already present in culture with fresh medium, and/or supplementing medium already present in culture with new medium. Fresh medium is medium that does not contain the one or more macromolecules or compounds to be introduced into at least one cell or medium that has not been in contact with cells to support their growth on cultivation. The skilled artisan can determine whether there is an advantage from or a need to remove and/or replenish, replace or supplement medium by monitoring cell growth and/or viability by techniques known in the art, such as cell counting (manual or automated), trypan blue exclusion, production of protein or other substance, alamar blue assay, presence or concentration of one or more metabolic products, cell adhesion, morphological appearance, analysis of spent medium, etc. One or a combination of monitoring techniques can be used to determine whether the medium needs to be to support growth, introduction of at least one macromolecule and/or cultivation after introduction of at least one macromolecule.

“Recombinant protein” refers to protein that is encoded by a nucleic acid that is introduced into a host cell. The host cell expresses the nucleic acid. “Protein” as used herein broadly refers to polymerized amino acids, e.g., peptides, polypeptides, proteins, lipoproteins, glycoproteins, etc.

The medium, methods, kits and compositions of the present invention are suitable for monolayer (adherent) or suspension culture, transfection, and cultivation of cells, and for expression of protein in cells in monolayer or suspension culture. Preferably, the medium, methods, kits and compositions of the present invention are for suspension culture, transfection, and cultivation of cells, and for expression of protein product in cells in suspension culture.

By “culture vessel” is meant any container, for example, a glass, plastic, or metal container, that can provide an aseptic environment for culturing cells.

The phrases “cell culture medium,” “tissue culture medium,” “growth medium,” “culture medium” (plural “media” in each case) and “medium formulation” refer to a nutritive solution for cultivating cells or tissues. These phrases can be used interchangeably.

The term “combining” refers to the mixing or admixing of ingredients.

An “inhibitor” refers to a compound (e.g. compounds described herein) that reduces activity when compared to a control, such as absence of the compound or a compound with known inactivity.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “down-regulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

### II. Medium

A major limitation of current systems used for the expansion of hematopoietic stem cells (HSCs) can be that ex vivo culture leads to expansion and differentiation of cells, at the expense of the most primitive pluripotent long-term stem cells (CD34⁺CD90⁺CD45RA⁻). This can limit the clinical application of ex vivo expanded HSC, since short-term progenitor cells only provide transient protection. The medium described herein increases the numbers of both CD34⁺ cells and CD34⁺CD90⁺CD45RA⁻ cells during culture to levels not seen in other media systems.

In an aspect, a growth medium is provided for culture of HSCs, including a basal medium and a supplement, with the medium and/or supplement including a histone acetyltransferase (HAT) inhibitor, a histone deacetylase (HDAC) inhibitor, and two or more of a lipid, an amino acid or amino acid derivative, an antioxidative agent, and an inorganic salt.

In some aspects, this disclosure relates to a medium for growth of hematopoietic stem cells (HSCs). The disclosure relates, in part, to a medium for cell culture comprising an inorganic salt selected from a barium salt, a cadmium salt, a copper salt, a magnesium salt, a selenite salt, a manganese salt, a nickel salt, a potassium salt, a calcium salt, a silver salt, a tin salt, a zirconium salt, a sodium salt, or combinations thereof; and a vitamin. In some embodiments, the medium is a chemically-defined medium. In some embodiments, the medium does not comprise protein. In some embodiments, the medium does not comprise serum. In some embodiments, the medium does not comprise an ingredient derived from an animal.

In some embodiments, a supplement is provided and used with the medium for growth of HSCs. In embodiments, the supplement is provided as a stock solution, for e× ample 2×, 5×, 10×, 20×, 25×, 30×, 40×, 50×, 60×, 70×, 75×, 80×, 90×, 100× stock solution (where “50×” indicates that the stock solution is a 50-fold solution, i.e., should be diluted 1:50 prior to use). In some embodiments, the supplement is a chemically-defined medium. In some embodiments, the supplement does not comprise protein. In some embodiments, the supplement does not comprise serum. In some embodiments, the supplement does not comprise an ingredient derived from an animal.

In some aspects, this disclosure relates to a kit with a basal media plus a supplement. In some embodiments, basal media is selected from Dulbecco's Modified Eagle Medium (DMEM); DMEM/F12, or a modified formulation thereof. Modified formulations of DMEM and DMEM/F12 include, without limitation, the basal media with or without HEPES, the basal media with or without phenol red, and the basal media with or without L-glutamine or a glutamine analog. In embodiments, basal media may be selected from: OpTmizer™ (Thermo Fisher Scientific); Dulbecco's Modified Eagle Medium (DMEM); Iscove's Modified Dulbecco's Medium (IMDM); DMEM/F12 phenol red-free, no HEPES; DMEM/F12 with phenol red, no HEPES; DMEM/F12 with HEPES and phenol red; DMEM/F12 with HEPES, no phenol red; DMEM/F12 with or without GLUTAMAX™ (Thermo Fisher Scientific); Advanced DMEM (Thermo Fisher Scientific); KnockOut™ DMEM/F12 (Thermo Fisher Scientific); or combinations thereof. In certain embodiments, each of the previously mentioned basal media may independently be excluded from a medium and/or supplement as described herein.

In embodiments, the kit includes the basal medium only. In embodiments, the kit includes the supplement only. In embodiments, the kit includes both the basal medium and the supplement. In embodiments, the kit includes the supplement as a stock solution, for example 2×, 5×, 10×, 20×, 25×, 30×, 40×, 50×, 60×, 70×, 75×, 80×, 90×, 100× stock solution. Where concentrations or ranges of concentrations for various components are recited herein, it is to be understood that the concentrations are provided as the 1× concentrations, i.e., the concentration to be used in the growth medium when expanding HSCs. Thus, the amount of a component recited herein may be 2-fold to 100-fold higher in the supplement, where the supplement is provided as a stock solution.

In some embodiments, the medium and/or supplement comprises an organic or inorganic salt selected from an aluminum salt, a barium salt, a cadmium salt, a copper salt, a magnesium salt, a manganese salt, a nickel salt, a potassium salt, a calcium salt, a silver salt, a tin salt, a zirconium salt, a sodium salt, or combinations thereof. Salts include those made with organic or inorganic anions including, without limitation: AgNO₃, AlCl₃, Ba(C₂H₃O₂)₂, CaCl₂, CdSO₄, CdCl₂, CoCl₂, Cr₂(SO₄)₃, CuCl₂, CuSO₄, FeSO₄, FeCl₂, FeCl₃, Fe(NO₃)₃, GeO₂, Na₂SeO₃, H₂SeO₃, KBr, KCl, KI, MgCl₂, MgSO₄, MnCl₂, NaCl, NaF, Na₂SiO₃, NaVO₃, Na₃VO₄, (NH₄)₆Mo₇O₂₄, Na₂HPO₄, NaH₂PO₄, NaHCO₃, NiSO₄, NiCl₂, Ni(NO₃)₂, RbCl, SnCl₂, ZnCl₂, ZnSO₄, ZrOCl₂, EDTA tetrasodium. In certain embodiments, each of the previously mentioned salts may independently be excluded from a medium and/or supplement as described herein.

In embodiments, the medium and/or supplement comprises an inorganic salt in a range of about 1×10⁻⁷ g/L to about 8.0 g/L. In embodiments, the medium and/or supplement comprises an inorganic salt in a range of about 5×10⁻⁷ g/L to about 8.0 g/L, about 1.5×10⁻⁶ g/L to about 8.0 g/L, about 1.4×10⁻⁶ g/L to about 8.0 g/L, or about 1.3×10⁻⁶ g/L to about 8.0 g/L. In embodiments, the medium and/or supplement comprises an inorganic salt in a range of about 1×10⁻⁷ g/L to about 7.8 g/L, about 1×10⁻⁷ g/L to about 7.6 g/L, about 1×10⁻⁷ g/L to about 7.4 g/L, about 1×10⁻⁷ g/L to about 7.2 g/L, or about 1×10⁻⁷ g/L to about 6.9 g/L. The concentration may be any value or subrange within the recited ranges, including endpoints.

In embodiments, the medium and/or supplement comprises an iron salt (e.g., FeSO₄, FeCl₂, FeCl₃, and/or Fe(NO₃)₃) in a range of about 1×10⁻⁶ g/L to about 5×10⁻³ g/L. In embodiments, the medium comprises an iron salt (e.g., FeSO₄, FeCl₂, FeCl₃, and/or Fe(NO₃)₃) in a range of about 5×10⁻⁶ g/L to about 5×10⁻³ g/L, about 1×10⁻⁵ g/L to about 5×10⁻³ g/L, about 5×10⁻⁵ g/L to about 5×10⁻³ g/L, or about 1×10⁻⁴ g/L to about 5×10⁻³ g/L. In embodiments, the medium comprises an iron salt (e.g., FeSO₄, FeCl₂, FeCl₃, and/or Fe(NO₃)₃) in a range of about 5×10⁻⁶ g/L to about 1×10⁻³ g/L, about 5×10⁻⁵ g/L to about 5×10⁻⁴ g/L, or about 5×10⁻⁵ g/L to about 1×10⁻⁴ g/L. In embodiments, the medium comprises an iron salt (e.g., FeSO₄, FeCl₂, FeCl₃, and/or Fe(NO₃)₃) in a range of about 1×10⁻⁵ g/L to about 5×10⁻⁴ g/L, 5×10⁻⁵ g/L to about 1×10⁻³ g/L, 1×10⁻⁵ g/L to about 5×10⁻³ g/L, 5×10⁻⁴ g/L to about 1×10⁻² g/L, or 1×10⁻⁴ g/L to about 5×10⁻² g/L. The concentration may be any value or subrange within the recited ranges, including endpoints.

In embodiments, the medium and/or supplement comprises a potassium salt (e.g., KBr, KCl, and/or KI) in a range of about 1×10⁻³ g/L to about 10 g/L. In embodiments, the medium comprises a potassium salt (e.g., KBr, KCl, and/or KI) in a range of about 5×10⁻³ g/L to about 10 g/L, about 1×10⁻² g/L to about 10 g/L, about 5×10⁻² g/L to about 10 g/L, or about 0.1 g/L to about 10 g/L. In embodiments, the medium comprises a potassium salt (e.g., KBr, KCl, and/or KI) in a range of about 5×10⁻³ g/L to about 5 g/L, about 5×10⁻³ g/L to about 1 g/L, or about 5×10⁻³ g/L to about 0.5 g/L. The concentration may be any value or subrange within the recited ranges, including endpoints.

In embodiments, the medium and/or supplement comprises a zinc salt (ZnCl₂ and/or ZnSO₄) in a range of about 1×10⁻⁵ g/L to about 5×10⁻² g/L. In embodiments, the medium comprises a zinc salt (ZnCl₂ and/or ZnSO₄) in a range of about 1×10⁻⁴ g/L to about 5×10⁻² g/L, about 1×10⁻³ g/L to about 5×10⁻² g/L, about 5×10⁻³ g/L to about 5×10⁻² g/L, or about 1×10⁻² g/L to about 5×10⁻² g/L. In embodiments, the medium comprises a zinc salt (ZnCl₂ and/or ZnSO₄) in a range of about 5×10⁻⁵ g/L to about 1×10⁻² g/L, about 5×10⁻⁵ g/L to about 5×10⁻³ g/L, or about 5×10⁻⁵ g/L to about 1×10⁻³ g/L. In embodiments, the medium comprises a zinc salt (ZnCl₂ and/or ZnSO₄) in a range of about 1×10⁻⁵ g/L to about 5×10⁻⁴ g/L, about 5×10⁻⁵ g/L to about 1×10⁻³ g/L, about 1×10⁻⁴ g/L to about 5×10⁻³ g/L, about 5×10⁻⁴ g/L to about 1×10⁻² g/L, or about 1×10⁻³ g/L to about 5×10⁻² g/L. The concentration may be any value or subrange within the recited ranges, including endpoints.

In embodiments, the medium and/or supplement comprises a magnesium salt (e.g., MgCl₂ and/or MgSO₄) in a range of about 0.001 g/L to about 1 g/L. In embodiments, the medium and/or supplement comprises a magnesium salt (e.g., MgCl₂ and/or MgSO₄) in a range of about 0.005 g/L to about 1 g/L, about 0.01 g/L to about 1 g/L, about 0.05 g/L to about 1 g/L, or about 0.1 g/L to about 1 g/L. In embodiments, the medium and/or supplement comprises a magnesium salt (e.g., MgCl₂ and/or MgSO₄) in a range of about 0.001 g/L to about 0.5 g/L, about 0.01 g/L to about 0.1 g/L, or about 0.01 g/L to about 0.05 g/L. In embodiments, the medium and/or supplement comprises a magnesium salt (e.g., MgCl₂ and/or MgSO₄) in a range of about 0.001 g/L to about 0.005 g/L, about 0.005 g/L to about 0.01 g/L, about 0.01 g/L to about 0.05 g/L, about 0.05 g/L to about 0.1 g/L, about 0.1 g/L to about 0.5 g/L, or about 0.5 g/L to about 1 g/L. The concentration may be any value or subrange within the recited ranges, including endpoints.

In some embodiments, the medium and/or supplement comprises a vitamin selected from para-aminobenzoic acid, vitamin B12, biotin, choline (e.g., choline chloride), tocopherol (e.g., tocopherol acetate), folic acid, ascorbic acid, inositol, nicotinic acid, niacinamide, pantothenic acid, calcium pantothenate, pyridoxine, pyroxidone, riboflavin, thiamine, or combinations thereof. In certain embodiments, each of the previously mentioned vitamins may independently be excluded from a medium and/or supplement as described herein. Each vitamin may be present in the medium and/or supplement in a range of about 1×10⁻⁶ g/L to about 5 g/L. In embodiments, the medium and/or supplement comprises the vitamin in a range of about 5×10⁻⁶ g/L to about 5 g/L, about 1×10⁻⁵ g/L to about 5 g/L, about 5×10⁻⁵ g/L to about 5 g/L, about 1×10⁻⁴ g/L to about 5 g/L, about 5×10⁻⁴ g/L to about 5 g/L, about 1×10⁻³ g/L to about 5 g/L, about 5×10⁻³ g/L to about 5 g/L, about 1×10⁻² g/L to about 5 g/L, about 5×10⁻² g/L to about 5 g/L, about 0.1 g/L to about 5 g/L, or about 1 g/L to about 5 g/L. In embodiments, the medium and/or supplement comprises the vitamin in a range of about 1×10⁻⁶ g/L to about 5×10⁻⁶ g/L, about 5×10⁻⁶ g/L to about 1×10⁻⁵ g/L, about 1×10⁻⁵ g/L to about 5×10⁻⁵ g/L, about 5×10⁻⁵ g/L to about 1×10⁻⁴ g/L, about 1×10⁻⁴ g/L to about 5×10⁻³ g/L, about 5×10⁻³ g/L to about 1×10⁻² g/L, about 1×10⁻² g/L to about 5×10⁻² g/L, about 5×10⁻² g/L to about 0.1 g/L, about 0.1 g/L to about 0.5 g/L, about 0.5 g/L to about 1 g/L, or about 1 g/L to about 5 g/L. The concentration may be any value or subrange within the recited ranges, including endpoints.

In some embodiments, the medium and/or supplement comprises a lipid, e.g., a fatty acid, selected from cholesterol, linoleic acid, linolenic acid, oleic acid, palmitic acid, arachidonic acid, palmitoleic acid, myristic acid, and combinations thereof. In certain embodiments, each of the previously mentioned lipids may independently be excluded from a medium and/or supplement as described herein. Each lipid may be present in the medium and/or supplement in a range of about 1×10⁻⁶ g/L to about 1×10⁻³ g/L. In embodiments, the medium and/or supplement comprises the lipid (e.g., fatty acid) in a range of about 5×10⁻⁶ g/L to about 1×10⁻³ g/L, about 1×10⁻⁵ g/L to about 1×10⁻³ g/L, about 5×10⁻⁵ g/L to about 1×10⁻³ g/L, about 1×10⁻³ g/L to about 1×10⁻³ g/L, or about 5×10⁻⁴ g/L to about 1×10⁻³ g/L. In embodiments, the medium and/or supplement comprises the lipid in a range of about 1×10⁻⁶ g/L to about 5×10⁻⁴ g/L, about 1×10⁻⁶ g/L to about 1×10⁻⁴ g/L, about 1×10⁻⁶ g/L to about 5×10⁻⁵ g/L, about 1×10⁻⁶ g/L to about 1×10⁻⁵ g/L, or about 1×10⁻⁶ g/L to about 5×10⁻⁶ g/L. The concentration may be any value or subrange within the recited ranges, including endpoints.

In some embodiments, the medium and/or supplement comprises an energy source selected from glucose, sucrose, pyruvate (e.g., sodium pyruvate), maltose, trehalose, or combinations thereof. In certain embodiments, each of the previously mentioned energy sources may independently be excluded from a medium and/or supplement as described herein. In embodiments, the medium and/or supplement comprises the energy source in a range between about 1×10⁻³ g/L and about 10 g/L. In embodiments, the medium and/or supplement comprises the energy source in a range between about 5×10⁻³ g/L and about 10 g/L, about 1×10⁻² g/L and about 10 g/L, about 5×10⁻² g/L and about 10 g/L, about 0.1 g/L and about 10 g/L, about 0.5 g/L and about 10 g/L, about 1 g/L and about 10 g/L, or about 5 g/L and about 10 g/L. In embodiments, the medium and/or supplement comprises the energy source in a range between about 1×10⁻³ g/L and about 5 g/L, about 1×10⁻³ g/L and about 1 g/L, about 1×10⁻³ g/L and about 0.5 g/L, about 1×10⁻³ g/L and about 0.1 g/L, about 1×10⁻³ g/L and about 5×10⁻² g/L, or about 1×10⁻³ g/L and about 1×10⁻² g/L. The concentration may be any value or subrange within the recited ranges, including endpoints.

In some embodiments, the medium and/or supplement comprises an amino acid selected from L-alanine, L-cysteine, L-aspartic acid, L-glutamic acid, L-phenylalanine, glycine, L-histidine, L-isoleucine, L-lysine, L-leucine, L-methionine, L-asparagine, pyrrolysine, L-proline, L-glutamine, L-arginine, L-serine, L-threonine, selenocysteine, L-valine, L-tryptophan, L-tyrosine, cystine, carnitine, levothyroxine, hydroxyproline, selenomethionine, taurine, citrulline, ornithine, or combinations thereof. In some embodiments, the amino acid is a stable analog of an amino acid (e.g., GLUTAMAX™, available from Thermo Fisher Scientific). In some embodiments, the medium and/or supplement comprises an amino acid derivative, e.g., N-acetyl-L-cysteine. In certain embodiments, each of the previously mentioned amino acids, analogs, and/or derivatives may independently be excluded from a medium and/or supplement as described herein. Each amino acid or amino acid derivative may be present in the medium and/or supplement in a range of about 1×10⁻⁴ g/L to about 10 g/L. In embodiments, the medium and/or supplement comprises the amino acid or amino acid derivative in a range of about 5×10⁻⁴ g/L to about 10 g/L, about 1×10⁻³ g/L to about 10 g/L, about 5×10⁻³ g/L to about 10 g/L, about 1×10⁻² g/L to about 10 g/L, about 5×10⁻² g/L to about 10 g/L, about 0.1 g/L to about 10 g/L, about 1 g/L to about 10 g/L, or about 5 g/L to about 10 g/L. In embodiments, the medium and/or supplement comprises the amino acid or amino acid derivative in a range of about 1×10⁻⁴ g/L to about 5 g/L, about 1×10⁻⁴ g/L to about 1 g/L, about 1×10⁻⁴ g/L to about 0.01 g/L, about 1×10⁻⁴ g/L to about 0.05 g/L, about 1×10⁻⁴ g/L to about 0.01 g/L, about 1×10⁻⁴ g/L to about 5×10⁻³ g/L, or about 1×10⁻⁴ g/L to about 1×10⁻³ g/L. In embodiments, the medium and/or supplement comprises the amino acid or amino acid derivative in a range of about 1×10⁻⁴ g/L to about 5×10⁻³ g/L, about 5×10⁻⁴ g/L to about 1×10⁻³ g/L, about 1×10⁻³ g/L to about 5×10⁻³ g/L, about 5×10⁻³ g/L to about 0.01 g/L, about 0.01 g/L to about 0.05 g/L, about 0.05 g/L to about 0.1 g/L, about 0.1 g/L to about 0.5 g/L, about 0.5 g/L to about 1 g/L, or about 1 g/L to about 5 g/L. The concentration may be any value or subrange within the recited ranges, including endpoints.

In some embodiments, the medium and/or supplement comprises serum albumin. In some embodiments, the serum albumin is human serum albumin. In some embodiments, the serum albumin is recombinant serum albumin (e.g., recombinant human serum albumin). In embodiments, the medium and/or supplement comprises serum albumin in a range between about 1 mL/L to about 900 mL/L. In embodiments, the medium and/or supplement comprises serum albumin in a range between about 1 mL/L to about 50 mL/L, about 5 mL/L to about 50 mL/L, about 10 mL/L to about 50 mL/L, or about 10 mL/L to about 20 mL/L. In embodiments, the medium and/or supplement comprises serum albumin in a range between about 1 mL/L to about 25 mL/L, or about 1 mL/L to about 20 mL/L. The concentration may be any value or subrange within the recited ranges, including endpoints. In certain embodiments, serum albumin may be excluded from a medium and/or supplement as described herein.

In some embodiments, the medium and/or supplement comprises insulin. In some embodiments, the insulin is human insulin. In some embodiments, the insulin is recombinant insulin (e.g., recombinant human insulin). In embodiments, the medium and/or supplement comprises insulin in a range between about 0.005 g/L to about 5 g/L. In embodiments, the medium and/or supplement comprises insulin in a range between about 0.01 g/L to about 0.5 g/L, about 0.05 g/L to about 0.5 g/L, about 0.1 g/L to about 0.5 g/L, or about 1 g/L to about 5 g/L. In embodiments, the medium and/or supplement comprises insulin in a range between about 0.005 g/L to about 0.1 g/L, about 0.005 g/L to about 0.05 g/L, or about 0.005 g/L to about 0.01 g/L. The concentration may be any value or subrange within the recited ranges, including endpoints. In certain embodiments, insulin may be excluded from a medium and/or supplement as described herein.

In some embodiments, the medium and/or supplement comprises transferrin. In some embodiments, the transferrin is human transferrin. In some embodiments, the transferrin is recombinant transferrin (e.g., recombinant human transferrin). In embodiments, the medium and/or supplement comprises transferrin in a range between about 0.005 g/L to about 10 g/L. In embodiments, the medium and/or supplement comprises transferrin in a range between about 0.01 g/L to about 0.5 g/L, about 0.05 g/L to about 0.5 g/L, about 0.1 g/L to about 0.5 g/L, about 1 g/L to about 5 g/L, about 5 g/L to about 10 g/L. In embodiments, the medium and/or supplement comprises transferrin in a range between about 0.005 g/L to about 0.1 g/L, about 0.005 g/L to about 0.05 g/L, or about 0.005 g/L to about 0.01 g/L. The concentration may be any value or subrange within the recited ranges, including endpoints. In certain embodiments, transferrin may be excluded from a medium and/or supplement as described herein.

In embodiments, the antioxidative agent is selected from polyphenols, ascorbate, and carotenoids. In embodiments, the polyphenol is selected from those found naturally in fruits, wines, and teas. In embodiments, the ascorbate is selected from ascorbate or ascorbic acid. In embodiments, the carotenoid is selected from beta-carotene, alpha-carotene and lycopene. In embodiments, the antioxidative agent is selected from DL lipoic acid, DL tocopherol acetate, and ascorbic acid.

In some embodiments, the medium and/or supplement comprises an additional ingredient selected from an emulsifier, a surfactant, an antioxidant, a buffer, a poloxamer, a metal binding compound, or combinations thereof. In some embodiments, the antioxidative agent is selected from polyphenols, ascorbate, and carotenoids. In embodiments, the polyphenol is selected from those found naturally in fruits, wines, and teas. In embodiments, the ascorbate is selected from ascorbate or ascorbic acid. In embodiments, the carotenoid is selected from beta-carotene, alpha-carotene and lycopene. In embodiments, the antioxidative agent is selected from DL lipoic Acid, DL tocopherol acetate, and ascorbic acid. In some embodiments, the medium and/or supplement comprises an additional ingredient selected from hypoxanthine or salt thereof, thymidine, polysorbate, ethanolamine, putrescine, spermine, sperimidine, EDTA, 2-mercaptoethanol, B-glycerophosphate, and hydrocortisone. In certain embodiments, each additional ingredient may individually be excluded from a medium and/or supplement as described herein. Each additional ingredient may be present in the medium and/or supplement in any amount, for example between about 5×10⁻⁶ g/L and about 1 g/L. In embodiments, the medium and/or supplement comprises the additional ingredient in a range between about 1×10⁻⁵ g/L and about 1 g/L, about 5×10⁻⁵ g/L and about 1 g/L, about 1×10⁻⁴ g/L and about 1 g/L, about 5×10⁻⁴ g/L and about 1 g/L, about 1×10⁻³ g/L and about 1 g/L, about 5×10⁻³ g/L and about 1 g/L, about 1×10⁻² and about 1 g/L, about 5×10⁻² and about 1 g/L, about 0.1 and about 1 g/L, or about 0.5 and about 1 g/L. In embodiments, the medium and/or supplement comprises the additional ingredient in a range between about 5×10⁻⁶ g/L and about 1×10⁻⁵ g/L, about 1×10⁻⁵ g/L and about 5×10⁻⁵ g/L, about 5×10⁻⁵ g/L and about 1×10⁻⁴ g/L, about 1×10⁻⁴ g/L and about 5×10⁻⁴ g/L, about 5×10⁻⁴ g/L and about 1×10⁻³ g/L, about 1×10⁻³ g/L and about 5×10⁻³ g/L, about 5×10⁻³ g/L and about 0.01 g/L, about 0.01 g/L and about 0.05 g/L, about 0.05 g/L and about 0.1 g/L, about 0.1 g/L and about 0.5 g/L, or about 0.5 g/L and about 1 g/L. The concentration may be any value or subrange within the recited ranges, including endpoints.

In some embodiments, the medium and/or supplement comprises a histone deacetylase (HDAC) inhibitor. In some embodiments, the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid. In some embodiments, the HDAC inhibitor is sodium butyrate, sodium phenylbutyrate, trichostatin A, or valproic acid. In certain embodiments, each of the previously mentioned HDAC inhibitors may independently be excluded from a medium and/or supplement as described herein.

The term “HDAC inhibitor” refers to a substance capable of detectably decreasing the expression or activity of histone deacetylase. The inhibitor can decrease expression or activity 10%, 20%, 30%, 40%, 50%, 60%, 70%, 80%, 90% or more in comparison to a control in the absence of the inhibitor. In certain instances, expression or activity is 1.5-fold, 2-fold, 3-fold, 4-fold, 5-fold, 10-fold or lower than the expression or activity in the absence of the inhibitor.

In certain embodiments, the HDAC inhibitor is present in the medium and/or supplement in any amount, for example a concentration between about 5×10⁻⁴ g/L and about 0.5 g/L. In embodiments, the HDAC inhibitor is present at a concentration between about 1×10⁻³ g/L and about 10 g/L, about 5×10⁻³ g/L and about 0.1 g/L, about 0.01 g/L and about 0.1 g/L, about 0.05 g/L and about 0.1 g/L, about 0.1 g/L and about 0.5 g/L, about 0.5 g/L and about 1 g/L, about 1 g/L and about 5 g/L, or about 5 g/L and about 10 g/L. In embodiments, the HDAC inhibitor is present at a concentration between about 5×10⁻⁴ g/L and about 0.1 g/L, about 5×10⁻⁴ g/L and about 0.05 g/L, about 5×10⁻⁴ g/L and about 0.01 g/L, about 5×10⁻⁴ g/L and about 5×10⁻³ g/L, or about 5×10⁻⁴ g/L and about 1×10⁻³ g/L. In embodiments, the HDAC inhibitor is present at a concentration between about 5×10⁻⁴ g/L and about 1×10⁻³ g/L, about 1×10⁻³ g/L and about 5×10⁻³ g/L, about 5×10⁻³ g/L and about 0.01 g/L, about 0.01 g/L and about 0.05 g/L, about 0.05 g/L and about 0.1 g/L, or about 0.1 g/L and about 0.5 g/L, about 0.5 g/L and about 1 g/L, about 1 g/L and about 5 g/L, or about 5 g/L and about 10 g/L. The concentration may be any value or subrange within the recited ranges, including endpoints.

In some embodiments, the medium and/or supplement comprises a histone acetyltransferase (HAT) inhibitor. In embodiments, the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin. In certain embodiments, each of the previously mentioned HAT inhibitors may independently be excluded from a medium and/or supplement as described herein.

The term “HAT inhibitor” refers to a substance capable of detectably decreasing the expression or activity of histone acetyltransferase. The inhibitor can decrease expression or activity 10%, 20%, 30%, 40%, 50%, 60%, 70%, 80%, 90% or more in comparison to a control in the absence of the inhibitor. In certain instances, expression or activity is 1.5-fold, 2-fold, 3-fold, 4-fold, 5-fold, 10-fold or lower than the expression or activity in the absence of the inhibitor.

In certain embodiments, the HAT inhibitor is present is present in the medium and/or supplement in any amount, for example a concentration of between 1×10⁻⁴ g/L and 1 g/L. In embodiments, the HAT inhibitor is present at a concentration between about 5×10⁻⁴ g/L and about 1 g/L, about 0.001 g/L and about 1 g/L, about 0.005 g/L and about 1 g/L, about 0.01 g/L and about 1 g/L, about 0.05 g/L and about 1 g/L, about 0.1 g/L and about 1 g/L, or about 0.5 g/L and about 1 g/L. In embodiments, the HAT inhibitor is present at a concentration between about 1×10⁻⁴ g/L and about 0.5 g/L, about 1×10⁻⁴ g/L and about 0.1 g/L, about 1×10⁻⁴ g/L and about 0.05 g/L, about 1×10⁻⁴ g/L and about 0.01 g/L, about 1×10⁻⁴ g/L and about 0.005 g/L, about 1×10⁻⁴ g/L and about 0.001 g/L. In embodiments, the HAT inhibitor is present at a concentration between about 1×10⁻⁴ g/L and about 5×10⁻⁴ g/L, about 5×10⁻⁴ g/L and about 0.001 g/L, about 0.001 g/L and about 0.005 g/L, about 0.005 g/L and about 0.01 g/L, about 0.01 g/L and about 0.05 g/L, about 0.05 g/L and about 0.01 g/L, about 0.01 g/L and about 0.05 g/L, about 0.05 g/L and about 0.1 g/L, or about 0.1 g/L and about 0.5 g/L. The concentration may be any value or subrange within the recited ranges, including endpoints.

In certain embodiments, the media comprises growth factors. Growth factors for various cell types are well known in the art. Growth factors that may be included in the growth medium described herein include, without limitation, interleukin 3 (IL-3), interleukin 6 (IL-6), stem cell factor (SCF), Fms-related tyrosine kinase 3 ligand (Flt3L), thrombopoietin (TPO), granulocyte colony-stimulating factor, and granulocyte-macrophage colony-stimulating factor. In certain embodiments, each of the previously mentioned growth factors may independently be excluded from a medium and/or supplement as described herein.

In embodiments, SCF is present in the medium and/or supplement at a concentration between about 0.0005 milligram per milliliter (mg/mL) and about 1 mg/mL. In embodiments, SCF is present in the medium and/or supplement at a concentration between about 0.0001 mg/mL and about 1 mg/mL, between about 0.005 mg/mL and about 1 mg/mL, about 0.01 mg/mL and about 1 mg/mL, about 0.05 mg/mL and about 1 mg/mL; about 0.1 mg/mL and about 1 mg/mL, or about 0.5 mg/mL and about 1 mg/mL. In embodiments, SCF is present in the medium and/or supplement at a concentration between about 0.0005 mg/mL and about 0.5 mg/mL, about 0.0005 mg/mL and about 0.1 mg/mL, about 0.0005 mg/mL and about 0.05 mg/mL; about 0.0005 mg/mL and about 0.01 mg/mL, about 0.0005 mg/mL and about 0.005 mg/mL, or about 0.0005 mg/mL and about 0.001 mg/mL. In embodiments, SCF is present in the medium and/or supplement at a concentration between about 0.0005 mg/mL and about 0.005 mg/mL, about 0.005 mg/mL and about 0.05 mg/mL, about 0.05 mg/mL and about 0.5 mg/mL; about 0.5 mg/mL and about 1 mg/mL.

In embodiments, Flt3L is present at a concentration between about 0.0005 mg/mL and about 1 mg/mL. In embodiments, Flt3L is present in the medium and/or supplement at a concentration between about 0.0001 mg/mL and about 1 mg/mL, between about 0.005 mg/mL and about 1 mg/mL, about 0.01 mg/mL and about 1 mg/mL, about 0.05 mg/mL and about 1 mg/mL; about 0.1 mg/mL and about 1 mg/mL, or about 0.5 mg/mL and about 1 mg/mL. In embodiments, Flt3L is present in the medium and/or supplement at a concentration between about 0.0005 mg/mL and about 0.5 mg/mL, about 0.0005 mg/mL and about 0.1 mg/mL, about 0.0005 mg/mL and about 0.05 mg/mL; about 0.0005 mg/mL and about 0.01 mg/mL, about 0.0005 mg/mL and about 0.005 mg/mL, or about 0.0005 mg/mL and about 0.001 mg/mL. In embodiments, Flt3L is present in the medium and/or supplement at a concentration between about 0.0005 mg/mL and about 0.005 mg/mL, about 0.005 mg/mL and about 0.05 mg/mL, about 0.05 mg/mL and about 0.5 mg/mL; about 0.5 mg/mL and about 1 mg/mL.

In embodiments, TPO is present at a concentration between about 0.0005 mg/mL and about 1 mg/mL. In embodiments, TPO is present in the medium and/or supplement at a concentration between about 0.0001 mg/mL and about 1 mg/mL, between about 0.005 mg/mL and about 1 mg/mL, about 0.01 mg/mL and about 1 mg/mL, about 0.05 mg/mL and about 1 mg/mL; about 0.1 mg/mL and about 1 mg/mL, or about 0.5 mg/mL and about 1 mg/mL. In embodiments, TPO is present in the medium and/or supplement at a concentration between about 0.0005 mg/mL and about 0.5 mg/mL, about 0.0005 mg/mL and about 0.1 mg/mL, about 0.0005 mg/mL and about 0.05 mg/mL; about 0.0005 mg/mL and about 0.01 mg/mL, about 0.0005 mg/mL and about 0.005 mg/mL, or about 0.0005 mg/mL and about 0.001 mg/mL. In embodiments, TPO is present in the medium and/or supplement at a concentration between about 0.0005 mg/mL and about 0.005 mg/mL, about 0.005 mg/mL and about 0.05 mg/mL, about 0.05 mg/mL and about 0.5 mg/mL; about 0.5 mg/mL and about 1 mg/mL.

In embodiments, IL-6 is present at a concentration between about 0.00005 mg/mL and about 0.1 mg/mL. In embodiments, IL-6 is present in the medium and/or supplement at a concentration between about 0.0001 mg/mL and about 0.1 mg/mL, between about 0.0005 mg/mL and about 0.1 mg/mL, about 0.001 mg/mL and about 0.1 mg/mL, about 0.005 mg/mL and about 0.1 mg/mL; about 0.01 mg/mL and about 0.1 mg/mL, or about 0.5 mg/mL and about 0.1 mg/mL. In embodiments, IL-6 is present in the medium and/or supplement at a concentration between about 0.00005 mg/mL and about 0.05 mg/mL, about 0.00005 mg/mL and about 0.01 mg/mL, about 0.00005 mg/mL and about 0.005 mg/mL; about 0.00005 mg/mL and about 0.001 mg/mL, about 0.00005 mg/mL and about 0.0005 mg/mL, or about 0.00005 mg/mL and about 0.0001 mg/mL. In embodiments, IL-6 is present in the medium and/or supplement at a concentration between about 0.00005 mg/mL and about 0.0005 mg/mL, about 0.0005 mg/mL and about 0.005 mg/mL, about 0.005 mg/mL and about 0.05 mg/mL; about 0.05 mg/mL and about 0.1 mg/mL.

In embodiments, IL-3 is present at a concentration between about 0.00005 mg/mL and about 0.1 mg/mL. In embodiments, IL-3 is present in the medium and/or supplement at a concentration between about 0.0001 mg/mL and about 0.1 mg/mL, between about 0.0005 mg/mL and about 0.1 mg/mL, about 0.001 mg/mL and about 0.1 mg/mL, about 0.005 mg/mL and about 0.1 mg/mL; about 0.01 mg/mL and about 0.1 mg/mL, or about 0.5 mg/mL and about 0.1 mg/mL. In embodiments, IL-3 is present in the medium and/or supplement at a concentration between about 0.00005 mg/mL and about 0.05 mg/mL, about 0.00005 mg/mL and about 0.01 mg/mL, about 0.00005 mg/mL and about 0.005 mg/mL; about 0.00005 mg/mL and about 0.001 mg/mL, about 0.00005 mg/mL and about 0.0005 mg/mL, or about 0.00005 mg/mL and about 0.0001 mg/mL. In embodiments, IL-3 is present in the medium and/or supplement at a concentration between about 0.00005 mg/mL and about 0.0005 mg/mL, about 0.0005 mg/mL and about 0.005 mg/mL, about 0.005 mg/mL and about 0.05 mg/mL; about 0.05 mg/mL and about 0.1 mg/mL.

In embodiments, granulocyte colony-stimulating factor (G-CSF) is present at a concentration between about 0.0005 mg/mL and about 1 mg/mL. In embodiments, G-CSF is present in the medium and/or supplement at a concentration between about 0.0001 mg/mL and about 1 mg/mL, between about 0.005 mg/mL and about 1 mg/mL, about 0.01 mg/mL and about 1 mg/mL, about 0.05 mg/mL and about 1 mg/mL; about 0.1 mg/mL and about 1 mg/mL, or about 0.5 mg/mL and about 1 mg/mL. In embodiments, G-CSF is present in the medium and/or supplement at a concentration between about 0.0005 mg/mL and about 0.5 mg/mL, about 0.0005 mg/mL and about 0.1 mg/mL, about 0.0005 mg/mL and about 0.05 mg/mL; about 0.0005 mg/mL and about 0.01 mg/mL, about 0.0005 mg/mL and about 0.005 mg/mL, or about 0.0005 mg/mL and about 0.001 mg/mL. In embodiments, G-CSF is present in the medium and/or supplement at a concentration between about 0.0005 mg/mL and about 0.005 mg/mL, about 0.005 mg/mL and about 0.05 mg/mL, about 0.05 mg/mL and about 0.5 mg/mL; about 0.5 mg/mL and about 1 mg/mL.

In embodiments, granulocyte-macrophage colony-stimulating factor (GM-CSF) is present at a concentration between about 0.00005 mg/mL and about 0.1 mg/mL. In embodiments, IL-6 is present in the medium and/or supplement at a concentration between about 0.0001 mg/mL and about 0.1 mg/mL, between about 0.0005 mg/mL and about 0.1 mg/mL, about 0.001 mg/mL and about 0.1 mg/mL, about 0.005 mg/mL and about 0.1 mg/mL; about 0.01 mg/mL and about 0.1 mg/mL, or about 0.5 mg/mL and about 0.1 mg/mL. In embodiments, GM-CSF is present in the medium and/or supplement at a concentration between about 0.00005 mg/mL and about 0.05 mg/mL, about 0.00005 mg/mL and about 0.01 mg/mL, about 0.00005 mg/mL and about 0.005 mg/mL; about 0.00005 mg/mL and about 0.001 mg/mL, about 0.00005 mg/mL and about 0.0005 mg/mL, or about 0.00005 mg/mL and about 0.0001 mg/mL. In embodiments, GM-CSF is present in the medium and/or supplement at a concentration between about 0.00005 mg/mL and about 0.0005 mg/mL, about 0.0005 mg/mL and about 0.005 mg/mL, about 0.005 mg/mL and about 0.05 mg/mL; about 0.05 mg/mL and about 0.1 mg/mL.

In some embodiments, a growth medium is provided for culture of HSCs, including a basal medium and a supplement, with the medium and/or supplement including a HAT inhibitor, a HDAC inhibitor, and two or more of a lipid, an amino acid or amino acid derivative, an antioxidative agent, and an inorganic salt, wherein the HAT inhibitor is present in the medium and/or supplement at a concentration of between about 0.001 g/L and about 1 g/L and the HDAC inhibitor is present in the medium and/or supplement at a concentration of between about 0.0005 g/L and 10 about g/L. In some embodiments, the HAT inhibitor is present in the medium or supplement at a concentration of between about 0.001 g/L and about 0.005 g/L and the HDAC inhibitor is present in the medium or supplement at a concentration of between about 0.01 g/L and about 0.1 g/L. In some embodiments, the HAT inhibitor is present in the medium or supplement at a concentration of between about 0.0001 g/L and about 0.01 g/L and the HDAC inhibitor is present in the medium or supplement at a concentration of between about 0.0005 g/L and about 0.1 g/L. In some embodiments, the HAT inhibitor is present in the medium and/or supplement at a concentration of between about 0.001 g/L and about 1 g/L and the HDAC inhibitor is present in the medium and/or supplement at a concentration of between about 1 g/L and about 5 g/L.

In some embodiments, the medium and/or supplement include a HAT inhibitor, a HDAC inhibitor and two or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin, and wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid. In some embodiments, the medium and/or supplement comprise a chalcone HAT inhibitor and a HDAC inhibitor selected from sodium butyrate, sodium phenylbutyrate, trichostatin A, and sodium valproate (VPA). In certain embodiments, the medium and/or supplement comprise a HAT inhibitor selected from garcinol, isogarcinol, xanthohumol, isoxanthohumol, 2-hydroxycalchone, 4-hydroxycalchone, yakuchinone A, and isoliquiritigenin, and a HDAC inhibitor selected from sodium butyrate, sodium phenylbutyrate, trichostatin A, and sodium valproate (VPA).

In some embodiments, the medium and/or supplement include a HAT inhibitor, a HDAC inhibitor and two or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the lipid is selected from cholesterol, linoleic acid, linolenic acid, oleic acid, palmitic acid, arachidonic acid, palmitoleic acid, myristic acid, and combinations thereof. In some embodiments, the medium and/or supplement include a HAT inhibitor, a HDAC inhibitor and two or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the antioxidative agent is selected from a polyphenol, ascorbate, a carotenoid, and combinations thereof. In some embodiments, the medium and/or supplement include a HAT inhibitor, a HDAC inhibitor and two or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the inorganic salt is selected from copper salt, a magnesium salt, a selenite salt, a potassium salt, a calcium salt, a zinc salt, an iron salt, a sodium salt, or combinations thereof.

In some embodiments, the medium and/or supplement include a HAT inhibitor, a HDAC inhibitor and three or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the lipid is selected from cholesterol, linoleic acid, linolenic acid, oleic acid, palmitic acid, arachidonic acid, palmitoleic acid, myristic acid, and combinations thereof. In some embodiments, the medium and/or supplement include a HAT inhibitor, a HDAC inhibitor and three or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the antioxidative agent is selected from a polyphenol, ascorbate, a carotenoid, and combinations thereof. In some embodiments, the medium and/or supplement include a HAT inhibitor, a HDAC inhibitor and three or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the inorganic salt is selected from copper salt, a magnesium salt, a selenite salt, a potassium salt, a calcium salt, a zinc salt, an iron salt, a sodium salt, or combinations thereof.

In some embodiments, the medium and/or supplement comprises a HAT inhibitor, a HDAC inhibitor, a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the lipid is selected from cholesterol, linoleic acid, linolenic acid, oleic acid, palmitic acid, arachidonic acid, palmitoleic acid, myristic acid, and combinations thereof. In some embodiments, the medium and/or supplement comprises a HAT inhibitor, a HDAC inhibitor, a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the antioxidative agent is selected from a polyphenol, ascorbate, a carotenoid, and combinations thereof. In some embodiments, the medium and/or supplement comprises a HAT inhibitor, a HDAC inhibitor, a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; and wherein the inorganic salt is selected from copper salt, a magnesium salt, a selenite salt, a potassium salt, a calcium salt, a zinc salt, an iron salt, a sodium salt, or combinations thereof.

In some embodiments, the medium and/or supplement comprises a HAT inhibitor, a HDAC inhibitor and two or more of a lipid, an amino acid or amino acid derivative, an antioxidative agent, and an inorganic salt, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol isoxanthohumol, 2-hydroxycalchone, 4-hydroxycalchone, yakuchinone A, and isoliquiritigenin; wherein the HDAC inhibitor is selected from apicidin, belinostat, CI-994, CRA-024781, curcumin, panobinostat, sodium butyrate, sodium phenylbutyrate, suberoylanilide hydroxamic acid, trichostatin A, sodium valproate (valproic acid; VPA), givinostat, MS-275, MGCD0103, and Scriptaid; wherein the lipid is selected from cholesterol, linoleic acid, linolenic acid, oleic acid, palmitic acid, arachidonic acid, palmitoleic acid, myristic acid, and combinations thereof; wherein the antioxidative agent is selected from a polyphenol, ascorbate, a carotenoid, and combinations thereof; and wherein the inorganic salt is selected from copper salt, a magnesium salt, a selenite salt, a potassium salt, a calcium salt, a zinc salt, an iron salt, a sodium salt, or combinations thereof.

In some embodiments, the medium and/or supplement comprises a HAT inhibitor, a HDAC inhibitor and two or more of a lipid, an amino acid or amino acid derivative, an antioxidative agent, and an inorganic salt, wherein the HAT inhibitor is selected from a chalcone such as garcinol, isogarcinol, xanthohumol isoxanthohumol, 2-hydroxycalchone, 4-hydroxycalchone, yakuchinone A, and isoliquiritigenin; wherein the HDAC inhibitor is selected from sodium butyrate, sodium phenylbutyrate, trichostatin A, and sodium valproate (VPA); wherein the lipid is selected from cholesterol, linoleic acid, linolenic acid, oleic acid, palmitic acid, arachidonic acid, palmitoleic acid, myristic acid, and combinations thereof; wherein the antioxidative agent is selected from a DL lipoic acid, DL tocopheral acetate and ascorbic acid; and combinations thereof; and wherein the inorganic salt is selected from copper salt, a magnesium salt, a selenite salt, a potassium salt, a calcium salt, a zinc salt, an iron salt, a sodium salt, or combinations thereof. In some embodiments, the HDAC inhibitor and HAT inhibitor are at a weight ration of 1:1 to 1:30 HDAC inhibitor: HAT inhibitor.

In some embodiments, the medium and/or supplement comprise a chalcone HAT inhibitor and a HDAC inhibitor selected from sodium butyrate, sodium phenylbutyrate, trichostatin A, and sodium valproate (VPA). In certain embodiments, the medium and/or supplement comprise a HAT inhibitor selected from garcinol, isogarcinol, xanthohumol, isoxanthohumol, 2-hydroxycalchone, 4-hydroxycalchone, yakuchinone A, and isoliquiritigenin, and a HDAC inhibitor selected from sodium butyrate, sodium phenylbutyrate, trichostatin A, and sodium valproate (VPA).

### III. Methods

In an aspect, the present disclosure relates to a method of expanding stem cells, the method including growing stem cells in a growth medium including a basal medium, a histone acetyltransferase (HAT) inhibitor, a histone deacetylase (HDAC) inhibitor, and two or more of a lipid, an amino acid or amino acid derivative, an antioxidative agent, and an inorganic salt.

In an aspect, the present disclosure relates to a method of expanding stem cells, the method including (i) adding a supplement including a histone acetyltransferase (HAT) inhibitor and a histone deacetylase (HDAC) inhibitor to a basal medium to form a growth medium, and (ii) growing stem cells in the growth medium, thereby expanding the stem cells.

In an aspect, the present disclosure relates to a method of expanding primary cells from a subject, the method including growing the primary cells in a growth medium including a basal medium, a histone acetyltransferase (HAT) inhibitor and a histone deacetylase (HDAC) inhibitor, and two or more of a lipid, an amino acid or amino acid derivative, an antioxidative agent, and an inorganic salt.

In an aspect, the present disclosure relates to a method of treating a subject in need of a therapy, the method including: (a) obtaining hematopoietic stem cells (HSCs); (b) expanding the HSCs in a growth medium including a basal medium, a histone acetyltransferase (HAT) inhibitor, and a histone deacetylase (HDAC) inhibitor, and at least two of a lipid, an amino acid or amino acid derivative, an antioxidative agent, and an inorganic salt; and (c) transferring the HSCs to the subject, thereby treating the subject.

Hematopoietic stem cells (HSCs), expanded in vitro according to any aspect of this disclosure may be used as a medicament. For example, HSCs expanded in a growth medium comprising a basal medium, a histone acetyltransferase (HAT) inhibitor, and a histone deacetylase (HDAC) inhibitor, and at least two of a lipid, an amino acid or amino acid derivative, an antioxidative agent, and an inorganic salt may be used as a medicament to treat a subject. The HSCs may be derived from the same subject that is to be treated and/or may be genetically modified prior to transferring the HSCs to the subject. Expanded HSCs may be used to treat a hematopoietic malignancy, cancer, an autoimmune disease, or a blood-based disease and/or after the subject has undergone chemotherapy. The subject may be human.

In an aspect, the present disclosure relates to a method for editing a genome in a stem cell (SC), the method including: (a) obtaining a SC that was expanded using the expansion methods provided herein (for example the methods of any one of claims 47 to 80); and (b) editing the genome of the stem cell. In embodiments, the genome is edited using one or more genome editing reagents selected from a zinc-finger nuclease (ZFN), transcription activator-like effector nuclease (TALEN), meganuclease, and a clustered regularly interspaced short palindromic repeat (CRISPR) associated protein, and (c) optionally expanding the edited cell in HSC expansion media as described herein for a period of time (e.g., 1 day, 2 days 3 days, 4 days, 5 days, 6 days, 1 week, 2 weeks, or longer, or any amount of time in between).

In an aspect, the present disclosure relates to a method for improving engraftment potential of a population of hematopoietic stem cells (HSCs), the method including: (a) obtaining a population of HSCs; and (b) expanding the population of HSCs using a medium as described herein, thereby improving engraftment potential of the population of HSCs.

In an aspect, the present disclosure relates to a method of treating a subject in need of a therapy, the method including: (a) obtaining hematopoietic stem cells (HSCs) that were expanded using a medium as described herein; and (b) transferring the HSCs to the subject, thereby treating the subject.

In an aspect, the present disclosure relates to a method of reprogramming a cell (e.g., a CD34+ cell, a PBMC) to an iPSC, the method including: (a) obtaining a cell to be re-programmed to an iPSC cell, (b) expanding the cell in HSC expansion media as described herein, (c) introducing the Yamanaka factors into the cell (see, for example Takahashi et al. (2007) Cell 131:861-872), (d) culturing the cell from (c) on a matrix in HSC expansion media for a first period of time, and (e) replacing the HSC expansion media with iPSC media, thereby reprogramming the cell. In some embodiments, (e) includes transitioning the cell from HSC expansion media to iPSC media. In some embodiments, the HSC expansion media is completely replaced with iPSC media at once. In some embodiments, the HSC expansion media is replaced with iPSC media in a stepwise fashion, until all of the HSC expansion media is replaced. In some embodiments, the reprogramming factors are introduced to the expanded CD34+ cells using a CytoTune™ iPS Reprogramming Kit (Thermo Fisher Scientific), such as CytoTune™-iPS 2.0 Sendai Reprogramming Kit or CTS™ CytoTune™-iPS 2.1 Sendai Reprogramming Kit.

### IV. Kits

In an aspect, the present disclosure relates to a kit including a basal medium and a supplement, the medium and/or supplement including a histone acetyltransferase (HAT) inhibitor, a histone deacetylase (HDAC) inhibitor, and two or more of a lipid, an amino acid or amino acid derivative, an antioxidative agent, and an inorganic salt. In some embodiments, the medium and/or supplement including a HAT inhibitor, a HDAC inhibitor, a lipid, an amino acid or amino acid derivative, an antioxidative agent, and an inorganic salt.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Hematopoietic Stem and Progenitor Cell Expansion System

Described is a media system that effectively expands both long term and short term human HSCs ex vivo. Human HSCs from different tissue sources such as cord blood, peripheral blood (e.g., mPB), and bone marrow also can be expanded. HSCs derived or expanded with this media may be used for any suitable use, including for example, biochemical, transcriptomic, epigenetic, and transplantation studies.

The use of a supplement in combination with a basal medium as described herein and referred to herein as “HSC Medium” to expand HSCs results in a higher proportion of CD34⁺CD90⁺CD45RA⁻ cells in the expanded cell population. The CD34⁺CD90⁺CD45RA⁻ cell population is relevant in the context of HSC transplantation, since this population is believed to be more important in establishing long term engraftment. Moreover, the addition of HAT inhibitor and HDAC inhibitor to competitor media did not result in a corresponding increase in the CD34⁺CD90⁺CD45RA⁻ subpopulation—indicating that the effect is not due solely to the presence of VPA/Garcinol, but rather to the supplement more generally.

Because this medium expands both short-term and long-term HSC, the patients who are transplanted with these expanded cells will not only have immune protection during the early phase of recovery (driven by short term CD34⁺ HSCs), but the long term cells (CD34⁺CD90⁺CD45RA⁻) would provide a suitable solution for transfusion-independent hematopoiesis. Ultimately, this is expected to increase positive health outcomes, reducing the duration of hospitalizations and health care costs per patient. The major types of diseases that may be targeted may include: cancer, autoimmune disorders, and blood disorders.

Other potential uses of this media system may include: gene therapy (e.g. CRISPR-Cas9), iPSC reprogramming (e.g., HSC to iPSC, PBMC to iPSC, and the like), transduction, differentiation (iPSC to HSC, HSC to immune cell types, HSC to erythrocytes, HSC to megakaryocytes), trans-differentiation (HSC to cardiomyocytes), small molecule screening for drug discovery, and disease modeling.

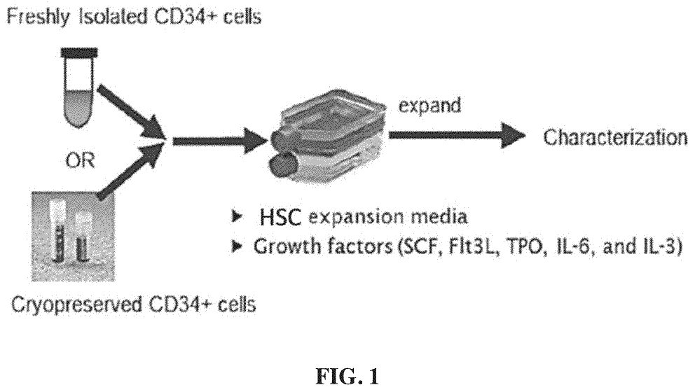

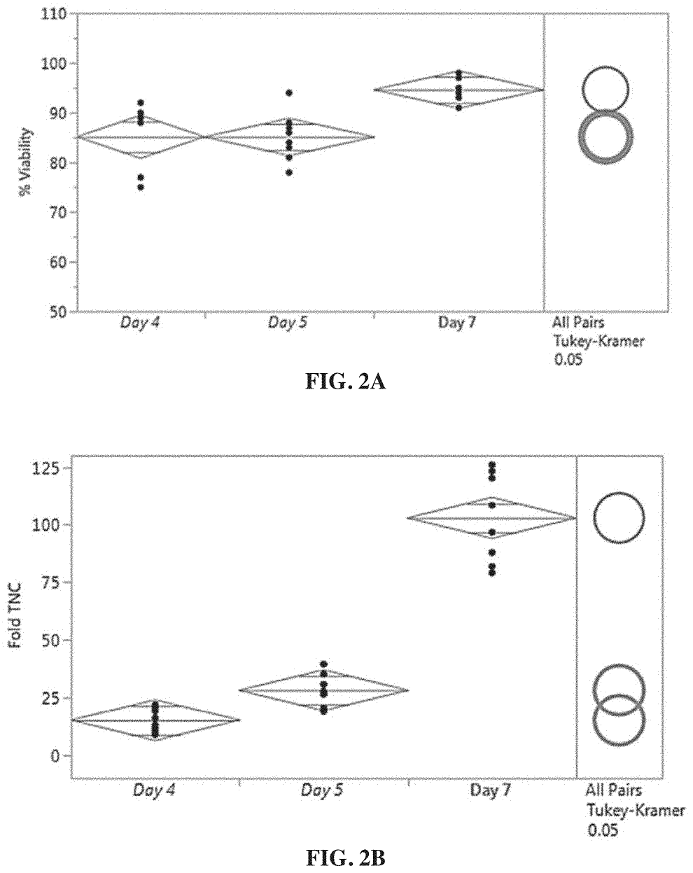

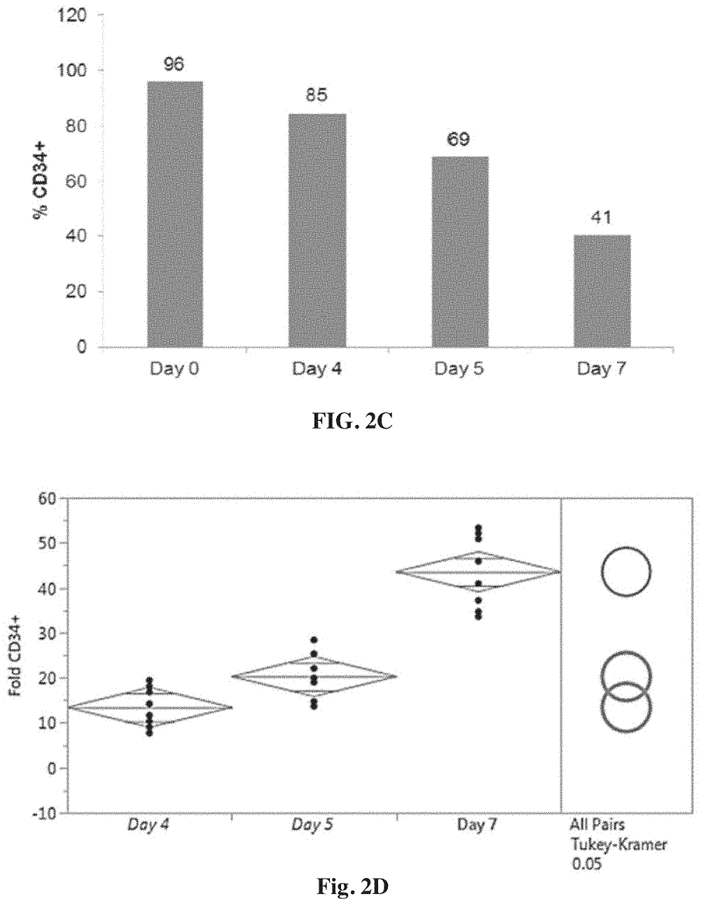

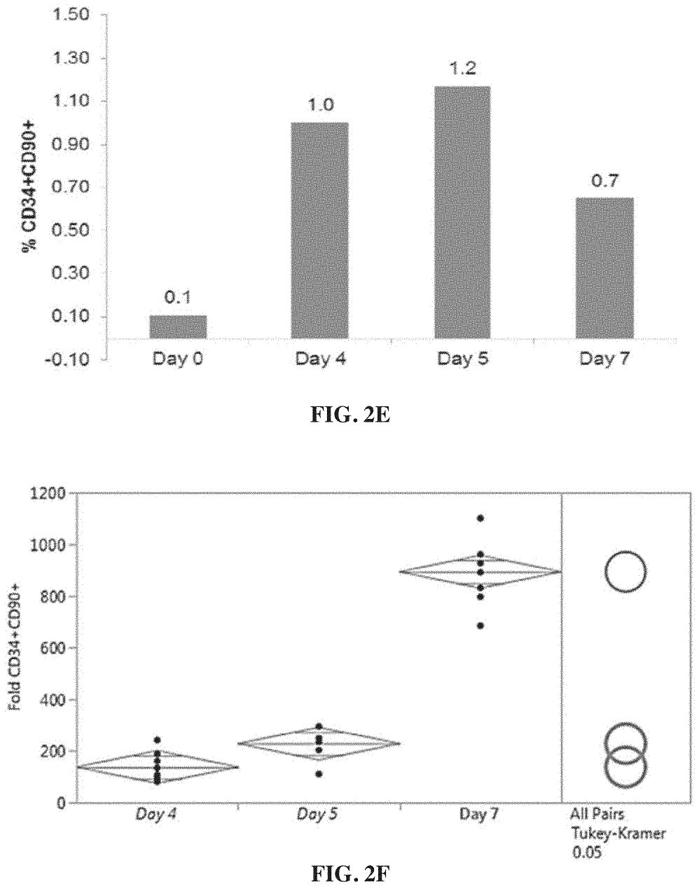

The basal medium used in the following examples was manufactured as a 1× formulation and stored at 4° C., while the supplement was a 50× formulation stored at −20° C. For cell expansion/culture, the thawed supplement was added to the basal medium, and specific growth factors were added to the complete media to promote HSC expansion. CD34⁺ cells from cord blood, bone marrow, or mobilized peripheral blood, as indicated below, were cultured in the presence of this media system for up to 14 days, in a 37° C. incubator. At Day 7 (or the indicated time point), cells were enumerated and assessed for phenotype by flow cytometry. The basal medium plus the supplement (at 1×) and growth factors (SCF, Flt3L, TPO, IL-6 and IL-3) enable the expansion of HSCs. See, e.g., FIG. 1.

In vitro expansion. Enriched CD34⁺ cells from three donors of human mobilized peripheral blood (mPB, AllCells) were cultured in the following serum free media: HSC Medium (as provided herein), and the commercially available Stem Cell Growth Medium (SCGM, CellGenix GmbH), STEMSPAN™ Animal Component Free (ACF, STEMCELL Technologies), and STEMPRO™ 34 (Thermo Fisher Scientific). All media were supplemented with 100 ng/mL SCF, 100 ng/mL FLT-3L, 100 ng/mL TPO, 50 ng/mL IL-3, and 20 ng/mL IL-6 (all provided by Thermo Fisher Scientific). HSC expansion was initiated by culturing 5×10³ CD34⁺ cells/mL in a 48 well plate for 7 days in a humidified 37° C., 5% CO₂ incubator. At day 7, total nucleated cell (TNC) number and % viability were assessed using a Countess II cell counter (Thermo Fisher Scientific), and cell phenotype was performed by flow cytometry.

Flow cytometry analysis. TNC were washed in phosphate-buffered saline (PBS) and incubated for 20 minutes at room temperature with LIVE/DEAD® Fixable Yellow stain in PBS. Cells were washed in flow cytometry staining buffer, and incubated with a cocktail of monoclonal antibodies against human cell surface antigens: CD34 PE-Cy7, CD90 APC, CD45RA Pacific Blue, for 30 minutes at 4° C. Cells were analyzed on a Attune NxT flow cytometer (Thermo Fisher Scientific), and FLOWJO® Software (version 7.6.5 Becton Dickinson).

Aldehyde dehydrogenase (ALDH) expression. Cells expanded for 7 days (as described above) were incubated with either ALDEFLUOR® or DEAB with ALDEFLUOR® for 45 minutes at 37° C. according to manufacturer's instructions (STEMCELL Technologies). Cells were then incubated with antibodies as described above, washed, and expression of ALDH by expanded cells was assessed by flow cytometry as described above. Gating for ALDH expression was determined by using DEAB controls.

### Example 2: Garcinol and VPA Synergistically Increase the Magnitude of HSC Expansion in HSC Medium Formulation

Effect of small molecules to medium performance. To assess the contribution of valproic acid (VPA) and garcinol to competitor media, the small molecules were added to SCGM and STEMSPAN™ ACF either in combination or individually at the final concentration present in HSC Medium. To address whether there is a reduction in performance of HSC Medium when the small molecules were excluded from the formulation, either VPA or garcinol were removed during manufacture. CD34⁺ mPB cells from three donors were cultured as described above (Example 1) in medium±VPA or garcinol, and compared with HSC medium, STEMSPAN™ ACF, SCGM or STEMSPAN™ ACF with STEMSPAN™ CD34⁺ Expansion Supplement (10×). At day 7, cells were assessed for TNC, % viability, CD34⁺, CD34⁺CD90⁺CD45RA⁻ cells as described above. To pool data points (CD34⁺ expansion, TNC expansion) from the three donors, all conditions were normalized to SCGM media the standard error calculated. For pooling of % CD34⁺ and % CD34⁺CD90⁺CD45RA⁻ data points in the three donors, the values were averaged and standard error calculated.

TABLE 1
Summary of experiments. Key: (+) is baseline,
(++) or greater improvement, (*) denotes
no data (the cells died when garcinol was added to ACF).

| | | HSC | | |
|---|---|---|---|---|
| Parameter | Small Molecules | Medium | SCGM | ACF |
| Fold CD34⁺ | +Garcinol | − | + | * |
| | +VPA | + | ++ | ++++ |
| | +Garcinol | ++++ | ++ | * |
| | +VPA | | | |
| % CD34⁺CD90⁺ | +Garcinol | + | + | * |
| | +VPA | ++++ | ++ | ++ |
| | +Garcinol | ++++ | ++ | * |
| | +VPA | | | |
| Fold TNC | +Garcinol | + | + | * |
| | +VPA | + | ++ | +++ |
| | +Garcinol | ++++ | + | * |
| | +VPA | | | |
| % CD34⁺ | +Garcinol | − | + | * |
| | +VPA | ++ | ++ | ++ |
| | +Garcinol | + | ++ | * |
| | +VPA | | | |

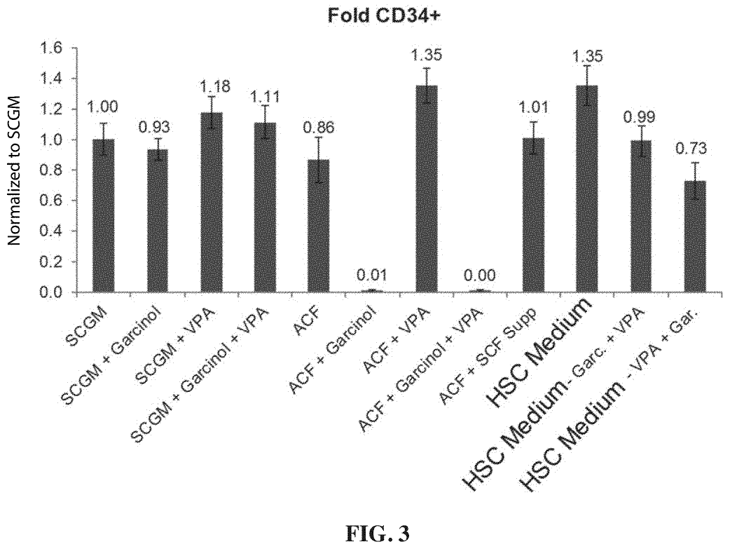

Garcinol and VPA in combination have a greater effect on CD34⁺ and CD34⁺CD90⁺ HSC expansion in HSC medium compared to competitor media. Addition of VPA alone to competitor media improved expansion of HSC in those media. In ACF, addition of VPA alone boosted performance to HSC Medium (with garcinol and VPA) levels. In HSC Medium Garcinol and VPA in combination work to improve performance. See FIG. 3.

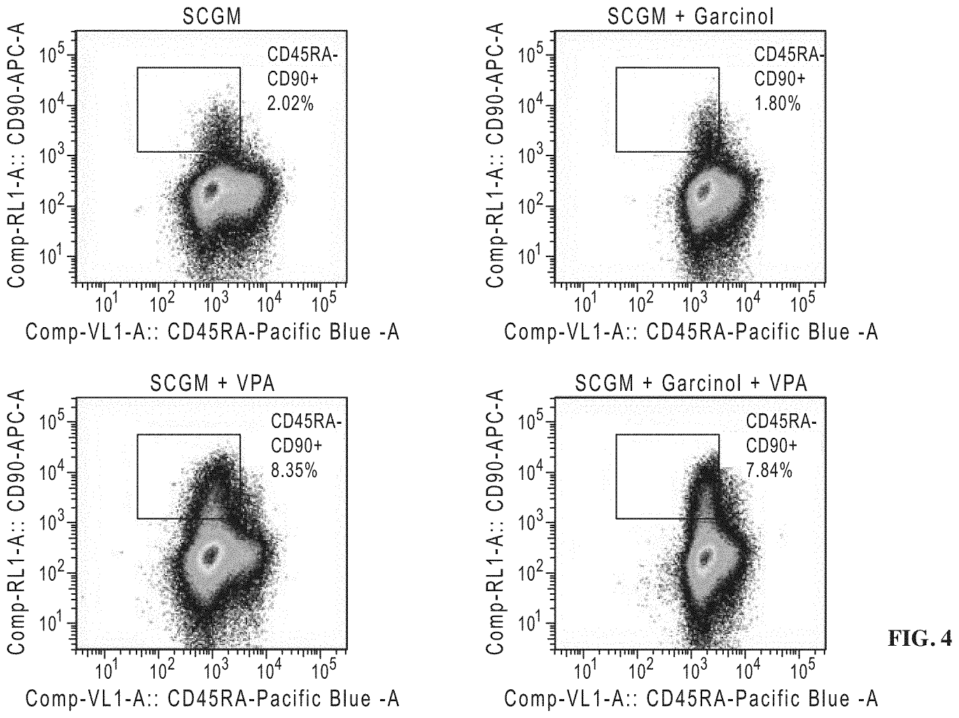

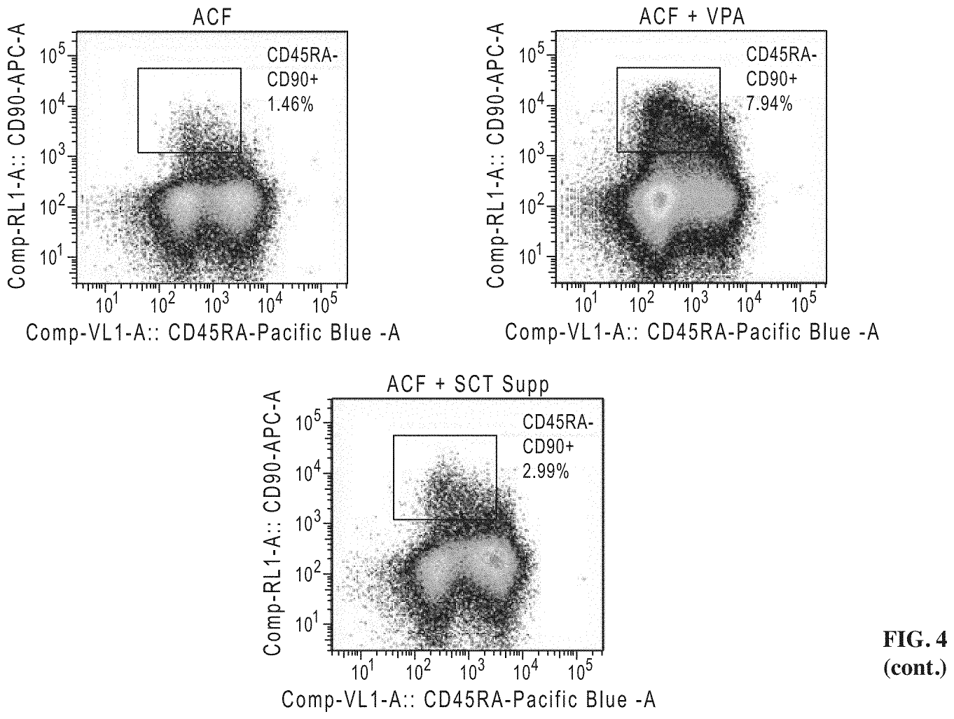

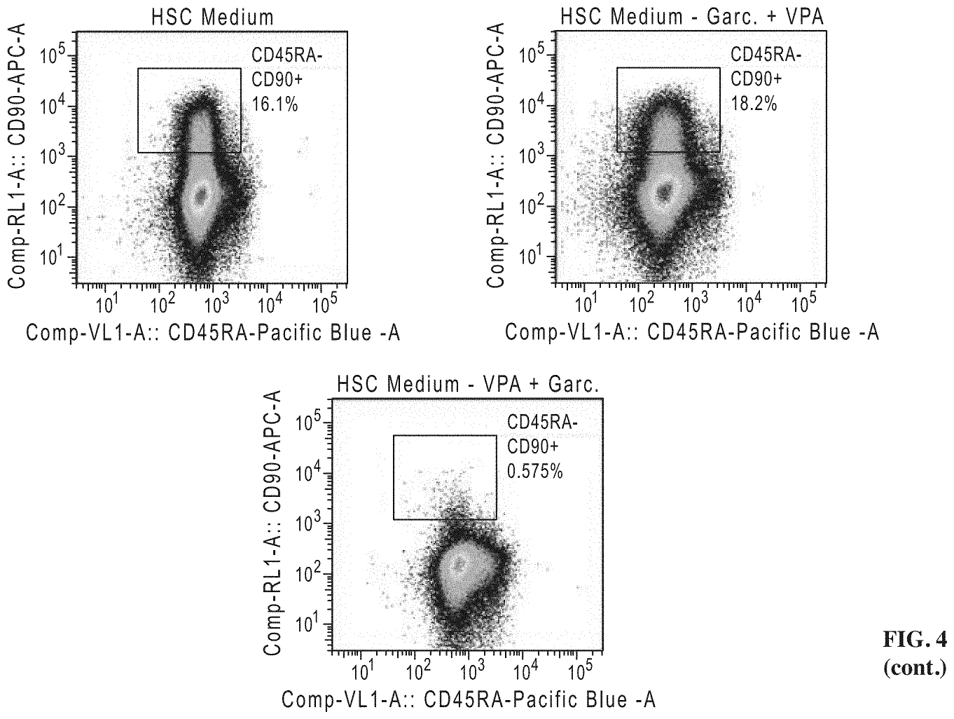

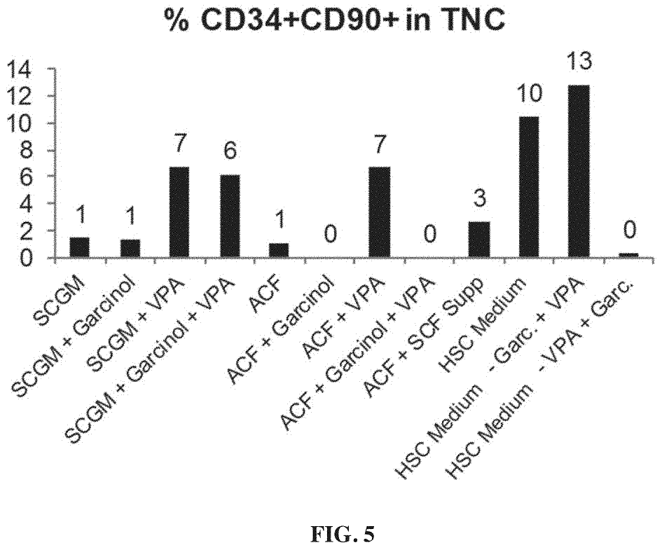

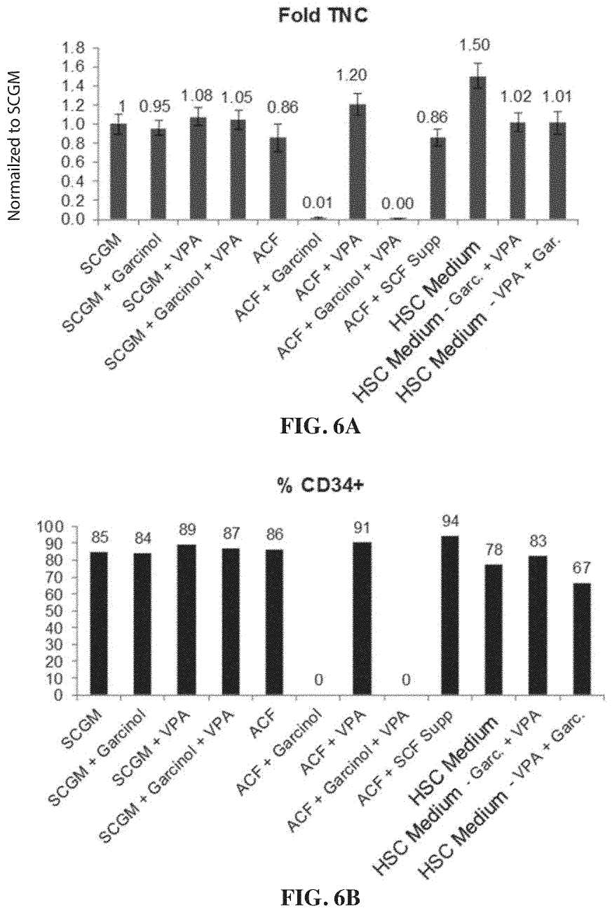

Addition of VPA alone to competitor media improved % CD34⁺CD90⁺CD45RA⁻ in HSC Medium Garcinol and VPA in combination improved % CD34⁺CD90⁺CD45RA⁻ to greater than competitor. See FIGS. 4 through 6B.

Conclusion. Garcinol and VPA do not work in combination when added to competitor media. In ACF, addition of VPA boosted performance to HSC Medium levels. In HSC Medium Garcinol and VPA in combination improved performance across TNC, CD34⁺, CD34⁺CD90⁺ populations.

### Example 3: HSC Medium

HSC Medium used in this example is a xeno-free, serum-free medium specifically formulated to support expansion of hematopoietic stem cells (HSCs). Phenotypes targeted for expansion are total CD34⁺ cell population, with an emphasis on enrichment of the CD34⁺CD90⁺CD45RA⁻ cell subset.

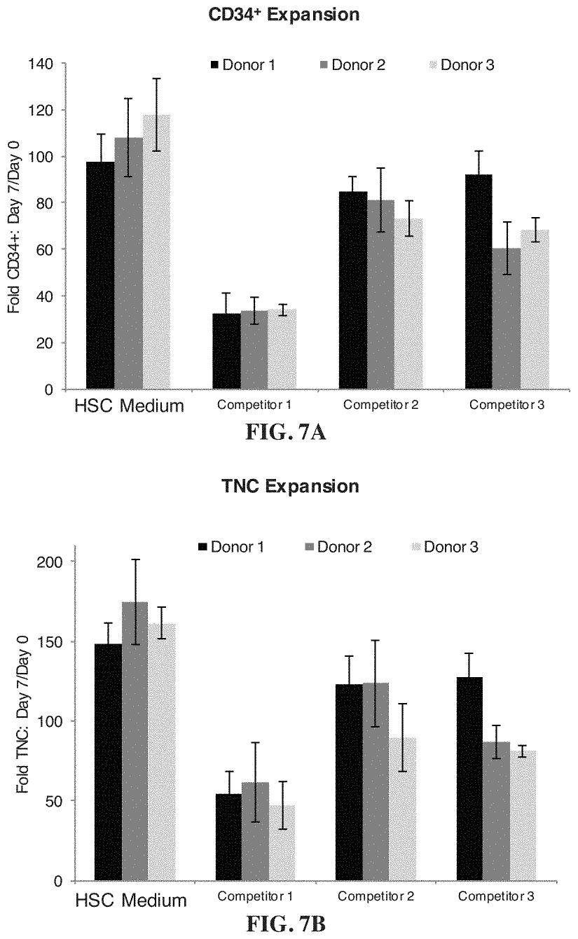

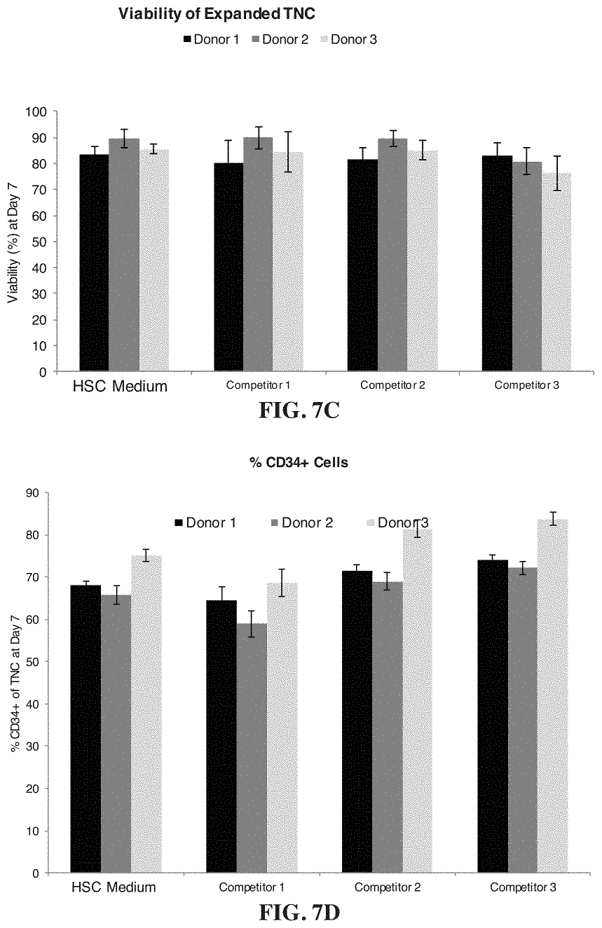

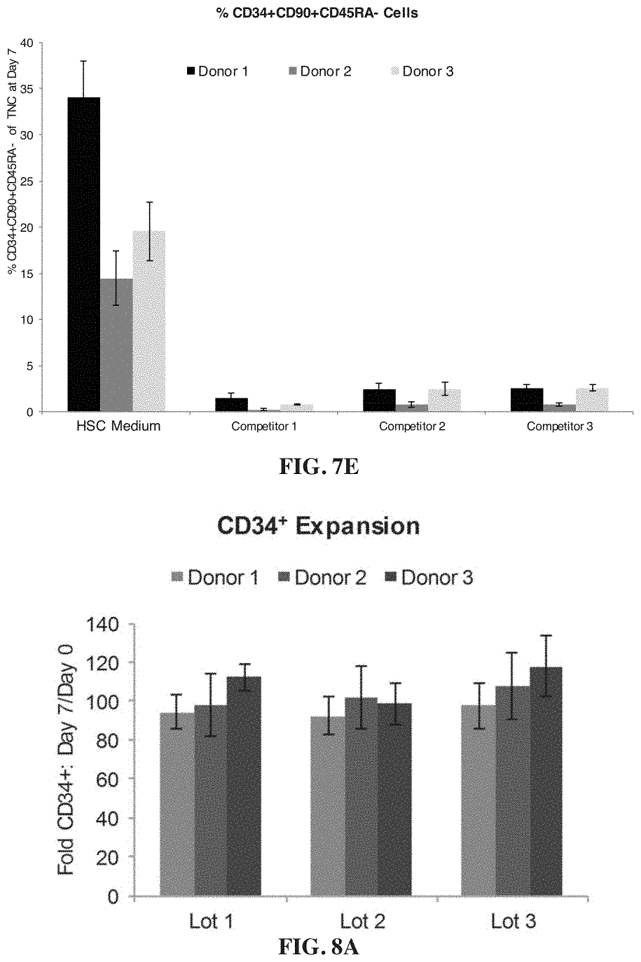

Superior expansion and enrichment. CD34⁺ cells and the CD34⁺CD90⁺CD45RA⁻ subset are found in low proportions in mobilized peripheral blood, bone marrow, as well as cord blood. They are important for proper hematopoietic and immune function, and are studied for a variety of reasons including transplantation biology, stem cell biology, and hematopoietic development. HSC Medium enables superior expansion of not only the total CD34⁺ compartment, but also enriches for CD34⁺CD90⁺CD45RA⁻ stem cells, providing the researcher with more cells to work with (see FIGS. 7-9).

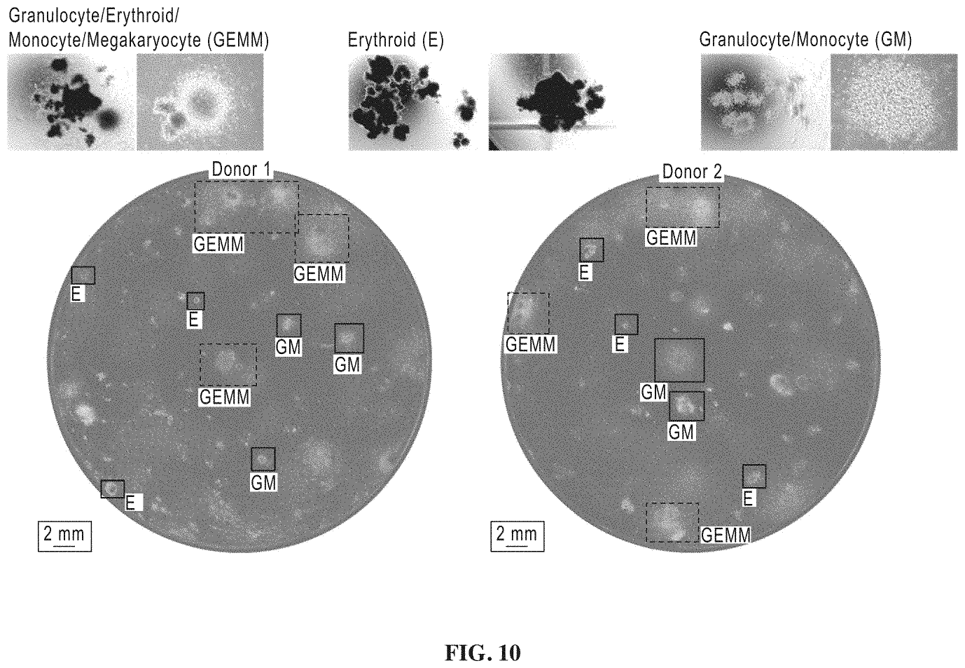

Maintenance of functionality. Functionality of expanded HSCs is critical. A frequently used in vitro assay for functional testing of HSCs is the colony forming unit (CFU) or colony forming cell (CFC) assay. The CFU assay was used to assess the proliferation and differentiation potential of HSCs by their ability to form colonies when seeded in a semi-solid medium. HSC Medium maintains in vitro differentiation capacity to granulocytic, monocytic, erythroid, and megakaryocytic lineages (see FIG. 10).

Total Nucleated Cell counts and % Viability. By utilizing the Countess II, we enumerated the total nucleated cells (TNC) and % Viability of the cells following expansion. The TNC represents all the different types of cells in culture (both HSC and differentiated cells). The % viability is important as some media conditions could adversely affect the survival of the cells.

Colony forming cell (CFC) assay. Expanded HSC must maintain their ability to differentiate into various blood cell lineages. In vivo, this is assessed via engraftment of HSC into immunodeficient mice. As these experiments typically take months to complete, a surrogate assay to determine differentiation potential is the colony forming cell (CFC) assay. In this assay, cells are seeded into a semi-solid MethoCult™ medium that contains growth factors such as erythropoietin, GM-CSF, G-CSF, IL-3, IL-6, SCF, which support the growth of progenitor cells such as erythrocytes, granulocyte, and macrophage progenitors. The different colonies are observed after a 14 day culture period.

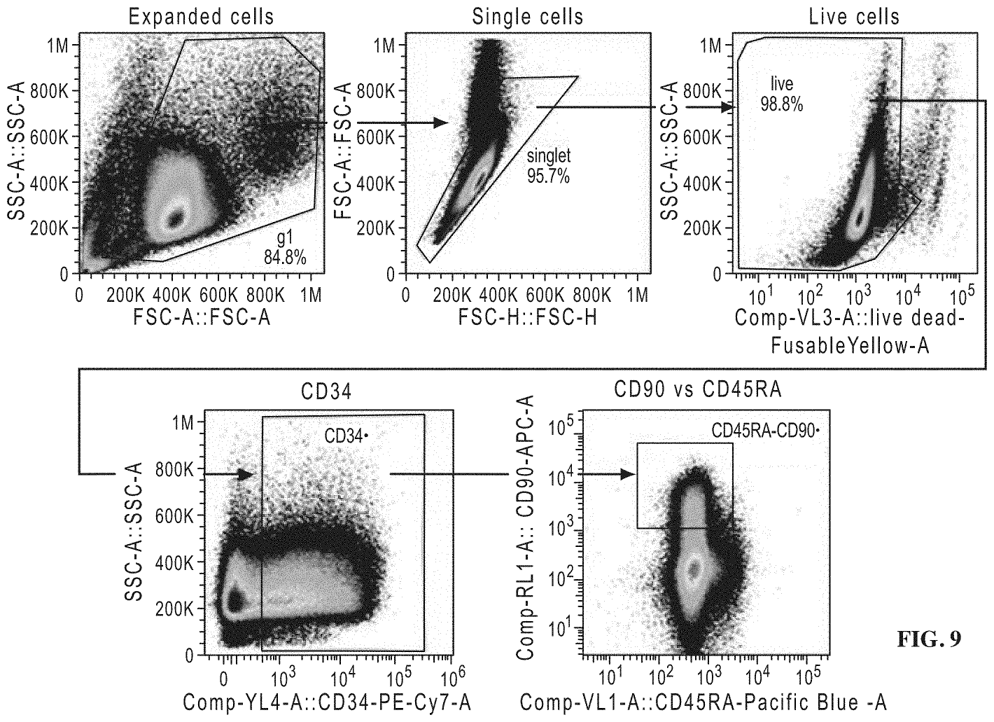

Competitive comparison of phenotypic performance data. Expansion of HSCs from human mobilized peripheral blood (mPB) in HSC Medium provides approximately 100-fold increase in CD34⁺ cells. Expansion of CD34⁺ mPB in HSC Medium led to significantly higher levels of expansion of CD34⁺ cells (FIG. 7A) and TNC (FIG. 7B), with cells demonstrating >80% viability (FIG. 7C). Expanded cells were >60% CD34⁺ (FIG. 7D), with significantly higher levels of CD34⁺CD90⁺CD45RA⁻ long term HSC (FIG. 7E). Percentages of CD34+ and CD34⁺CD90⁺CD45RA⁻ shown are from the total live TNC population. Three human single donor purified CD34⁺ mPB were cultured in HSC Medium (HSC Basal and 50× Supplement) or three commercial media, all supplemented with growth factors (SCF, Flt3L, TPO, IL-3, and IL-6). Cells were cultured for 7 days, following which total nucleated cells (TNC) and % viability were determined using a Countess II, and phenotype assessed by flow cytometry as described below and in FIG. 9. Error bars denote standard deviation.

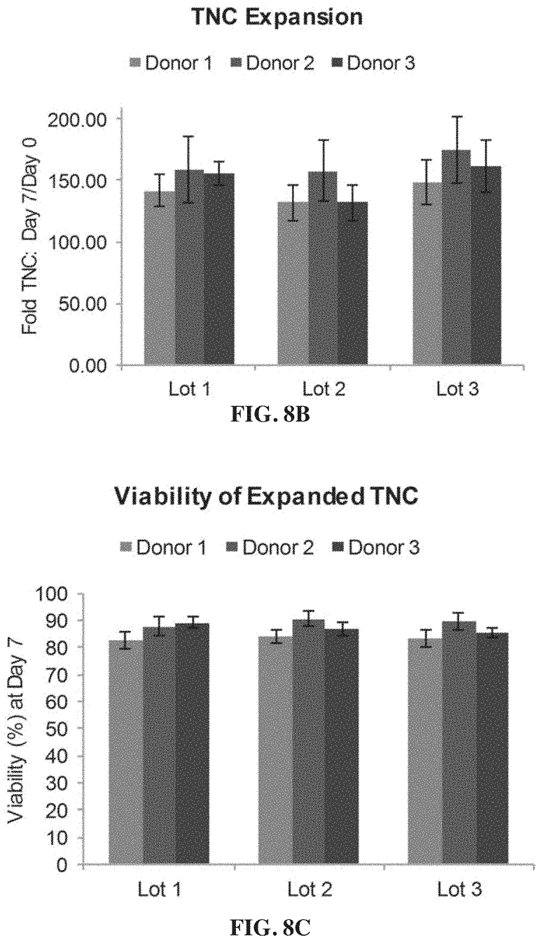

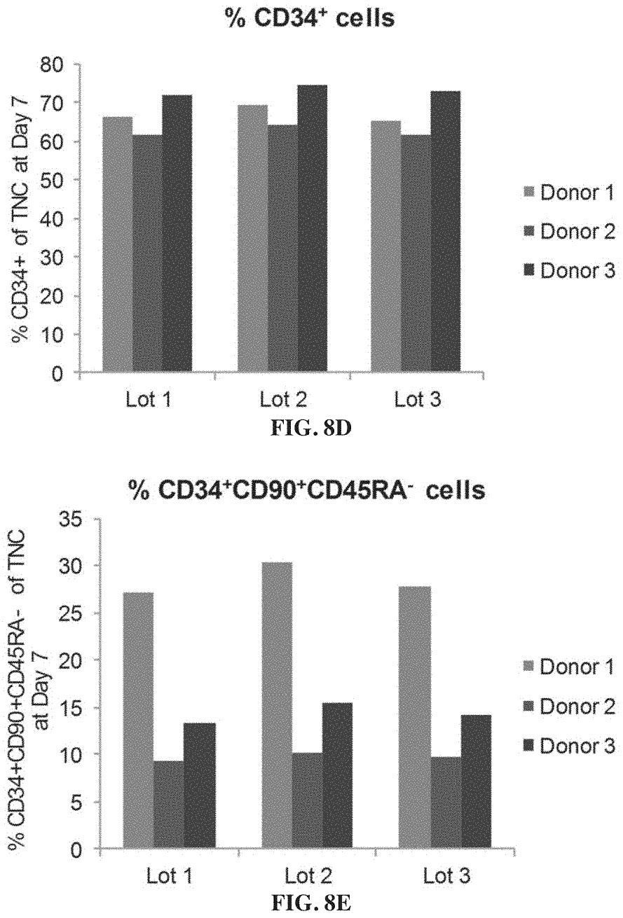

Lot to lot phenotypic performance data. HSC Medium demonstrates Consistent Lot-to-Lot Performance. CD34⁺ mPB expanded in three different lots of HSC Medium demonstrate equivalent levels of CD34⁺ cell expansion (FIG. 8A), TNC expansion (FIG. 8B), % viability (FIG. 8C), % CD34⁺ (FIG. 8D), and % CD34⁺CD90⁺CD45RA⁻ long term HSC (FIG. 8E). Three human single donor purified CD34⁺ mPB were cultured in three different lots of HSC Medium (HSC Basal and 50× Supplement), all supplemented with growth factors. Cells were cultured for 7 days, following which total nucleated cells (TNC) and % viability were determined using a Countess II, and phenotype assessed as described in FIG. 9. Error bars denote standard deviation.

Phenotypic analysis gating strategy: Phenotypic Characterization of Expanded HSC Cultured in HSC Medium. Purified CD34⁺ from mPB were cultured in HSC Medium containing growth factors. Cells were cultured for 7 days, then assessed by flow cytometry for expression of CD34, CD90, and CD45RA. Doublets and dead cells were excluded from analysis, and gates identifying CD34⁺ cells and CD90⁺CD45RA⁻ cells were demarcated based on fluorescent minus one (FMO) controls.

CFU assay data. CD34⁺ cells expanded in HSC Medium maintain in vitro differentiation capacity. CD34⁺ mPB expanded in HSC Medium were able to differentiate into Granulocyte/Erythroid/Monocyte/Megakaryocyte (GEMM), Erythroid (E), and Granulocyte/Monocyte (GM) colony forming cells during a 14 day culture period. Two human single donor purified CD34⁺ mPB were cultured for 7 days in HSC Medium, supplemented with growth factors. Subsequently, expanded TNC were cultured for another 14 days in semi-solid medium to assess for Colony forming cells (CFC). Images show examples of colonies identified.

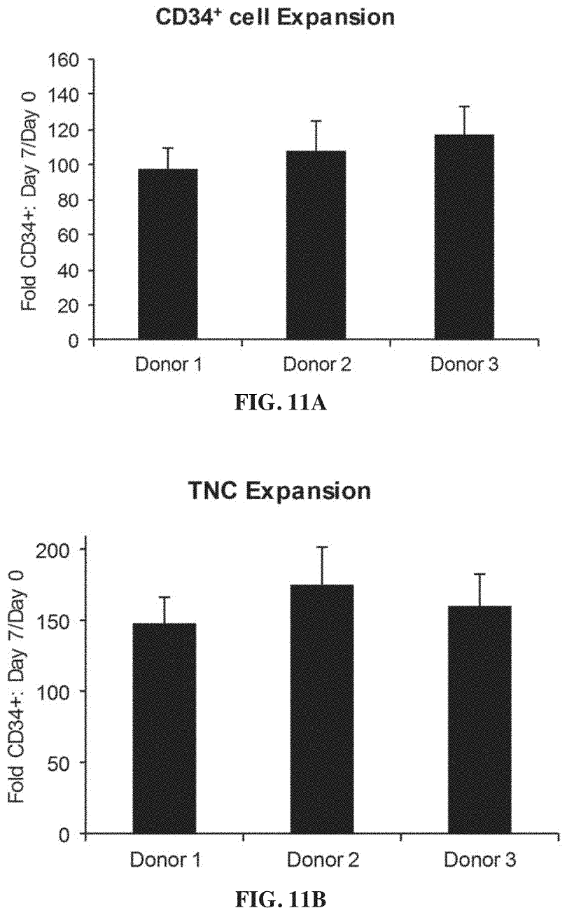

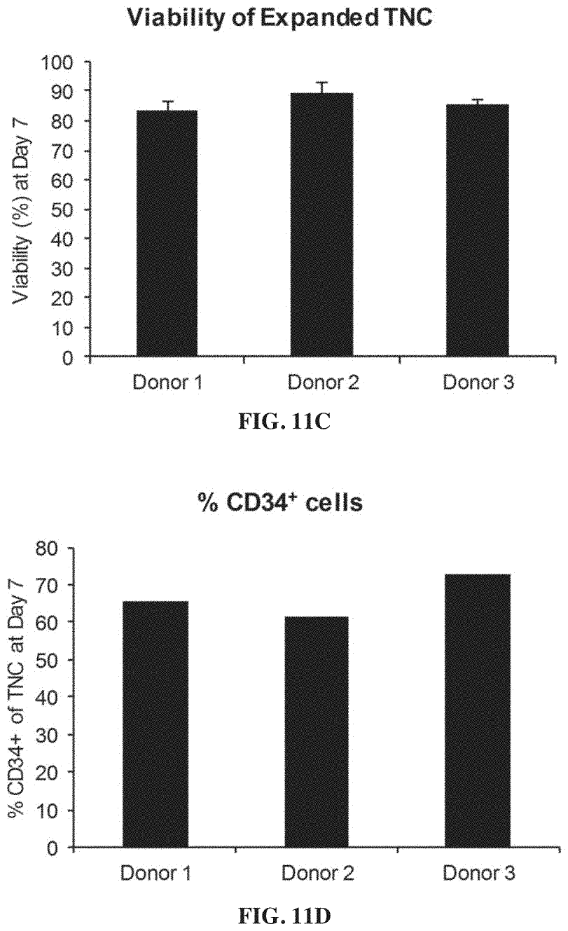

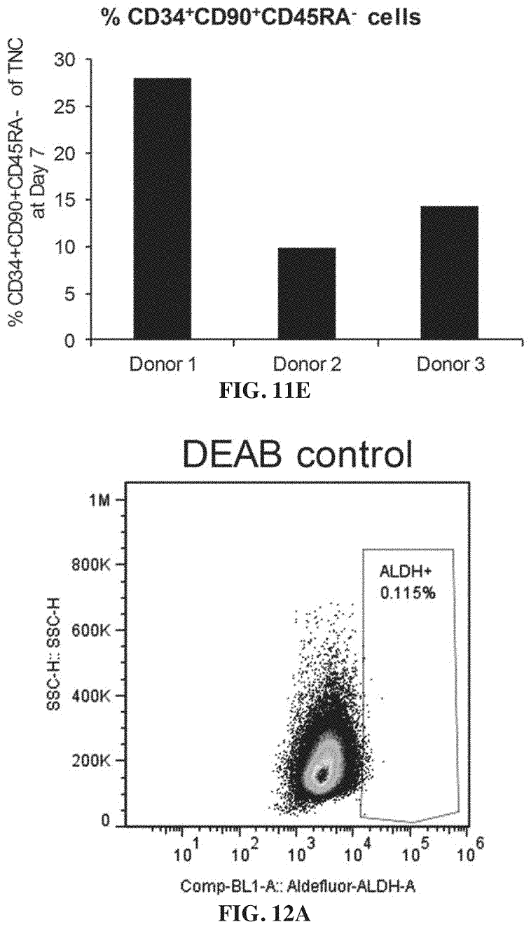

HSC Medium Expands Single donor human CD34⁺ mPB cells. All three human single donor CD34⁺ mPB that were expanded in HSC Medium demonstrated equivalent levels of expansion in CD34⁺ cells (FIG. 11A), TNC (FIG. 11B), % viability (FIG. 11C), % CD34+(FIG. 11D). Notably, the level of expansion of CD34⁺CD90⁺CD45RA⁻ long term HSC (FIG. 11E) varied amongst donors. Three human single donor purified CD34⁺ mPB were cultured in HSC Medium, supplemented with growth factors. Cells were cultured for 7 days, following which total nucleated cells (TNC) and % viability were determined using a Countess II, and phenotype assessed as described in FIG. 9. Error bars denote standard deviation within sample replicates.

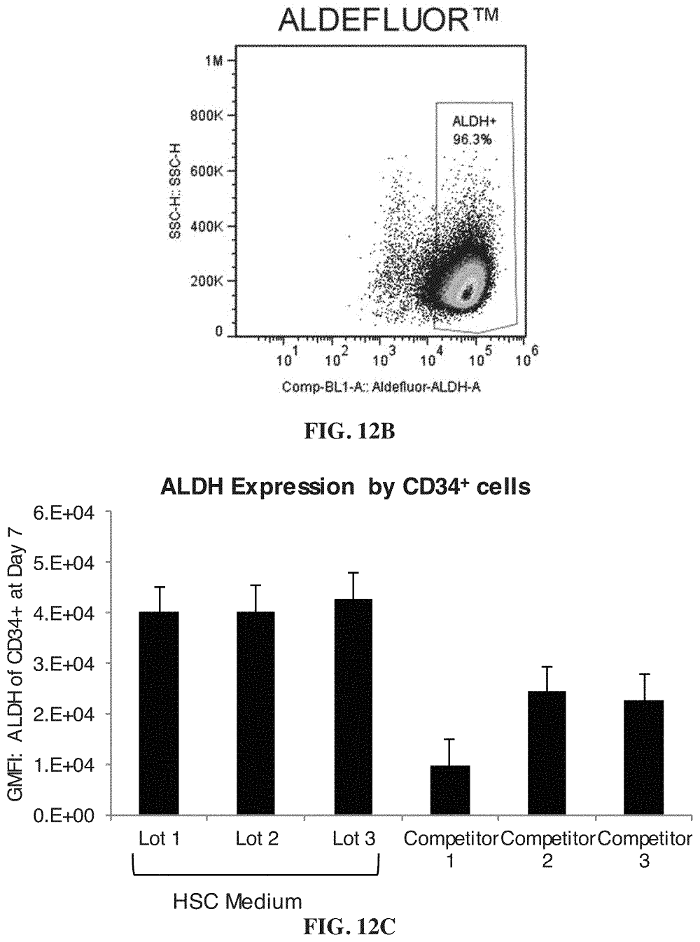

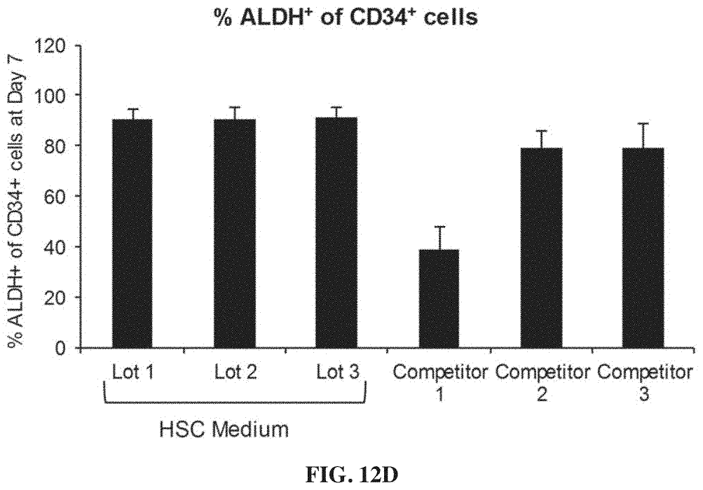

CD34⁺ cells Expanded in HSC Medium express highest ALDH levels. CD34⁺ mPB expanded in HSC Medium were assessed for expression of Aldehyde dehydrogenase (ALDH). Gating was used to demonstrate identification of cells incubated in (FIG. 12A) control DEAB with no ALDH expression, compared to (FIG. 12B) ALDEFLUOR™ positive cell populations. Expansion of CD34⁺ in HSC Medium demonstrated (FIG. 12C) highest ALDH expression on a per-cell basis (Geometric Mean Fluorescent Intensity), and (FIG. 12D) highest % of CD34⁺ cells staining positive for ALDH. Consistency in ALDH expression by expanded cells was observed between HSC Medium lots. Three human single donor purified CD34⁺ mPB were cultured in three lots of HSC Medium or three commercial media, all supplemented with growth factors. Cells were cultured for 7 days, following which total nucleated cells (TNC) were incubated in either DEAB or ALDEFLUOR™, according to manufacturer's instructions. Cells were stained with antibodies to identify CD34⁺ cells, and analyzed for ALDH expression. Data displayed were pooled from the three single donor mPB cells. Error bars denote standard error.

### Example 4: Genetic Engineering of HSCs Expanded in HSC Expansion Media

CD34+ cells Expanded in HSC Medium are capable of being genetically engineered using CRISPR/Cas9. 1×10⁶ purified CD34+ cells from mobilized peripheral blood (mPB) from a single donors was individually cultured in HSC Medium described herein, supplemented with 100 ng/mL SCF, 100 ng/mL FLT-3L, 100 ng/mL TPO, 50 ng/mL IL-3, and 20 ng/mL IL-6 (all provided by Thermo Fisher Scientific). Cells were cultured for 2 days in a 37° C. incubator, 5% CO₂. At Day 2, 5×10⁴ cells from each donor were transfected using the NEON™ cell transfection device (Thermo Fisher Scientific), according to manufacturer's instructions. Specifically, cells were transfected with 1.2 μg TRUECUT™ Cas9 Protein V2 (Thermo Fisher Scientific), 300 ng guide RNA, and 100 ng/ml, 200 ng/ml, or 500 ng/ml 1.4 kb GFP donor DNA. Following transfection, the cells were cultured in STEMPRO™ HSC Expansion Medium with SCF, Flt3L, TPO, IL-3 and IL-6 for 3 days. The efficiency of insertion of the GFP donor DNA was measured by ATTUNE™ NxT Acoustic Focusing Cytometer (Thermo Fisher Scientific) at 72 hours post transfection. Using the un-transfected cells to set the gate, the percentages of GFP+ cells were determined. To measure the pluripotency of HSC, the un-transfected and transfected cells were stained with both CD34-PE Cy7 (Thermo Fisher Scientific, 25-0349-42) and CD90-APC (Thermo Fisher Scientific, 17-0909-42) antibodies. Gate settings and compensations of various fluorescence channels were carried out using the un-transfected cells. The CD34 and CD90 staining of GFP-cell population was used to compare to that of GFP+ cell population. The GFP+ cells were further isolated from transfected cells using the BioRad s3e cell sorter.

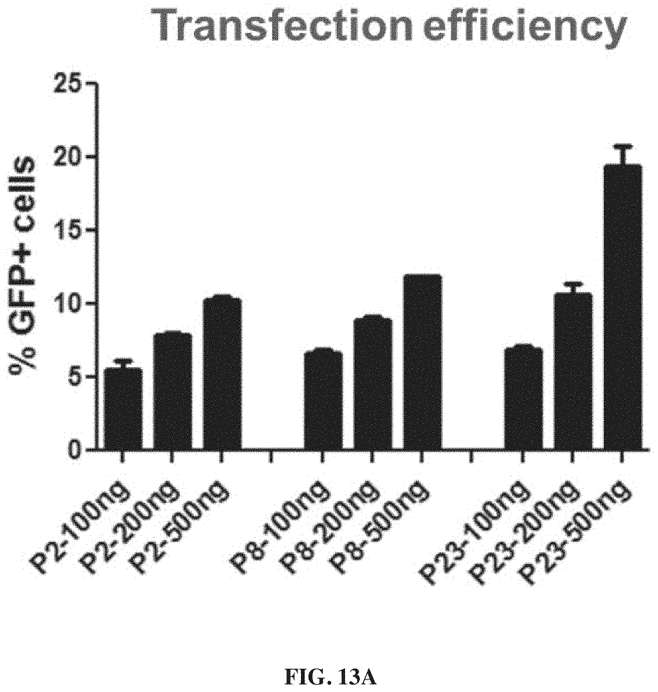

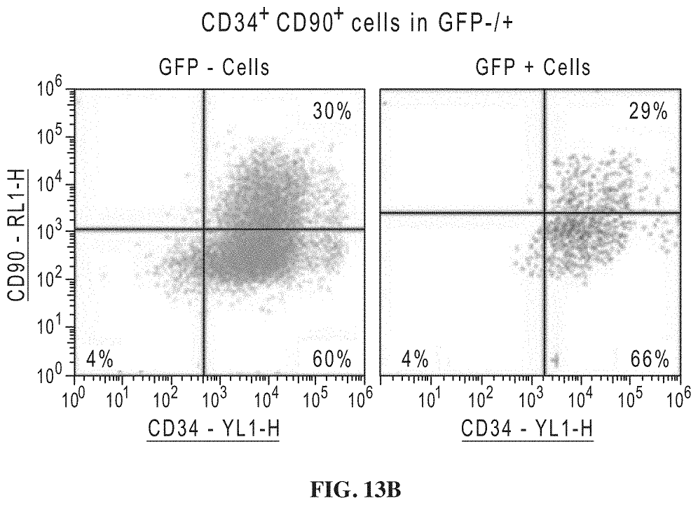

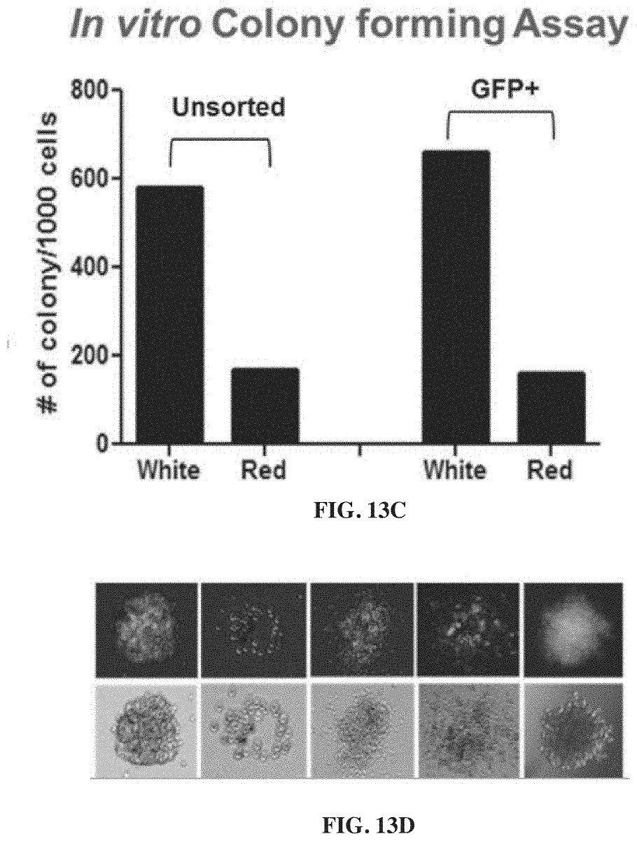

GFP+ and GFP−-sorted cells were analyzed by flow cytometry for CD34 and CD90 using an Attune NxT flow cytometer (Thermo Fisher Scientific). The bars indicate the mean value of the 3 individual donors. (FIG. 13B). Transfected GFP+ cells exhibited a similar CD34, CD90 profile when compared to the un-transfected GFP− cells, demonstrating that expansion of CD34+ cells in HSC expansion media as described herein does not negatively impact the ability to genetically modify the cells. GFP+ genetically engineered cells displayed similar ability to differentiate compared to non-modified cells, using a colony forming assay (FIG. 13C).

### Example 5: STEMPRO™ HSC Enables Reprogramming of CD34+ Cells into iPSC with CTS CytoTune 2.1

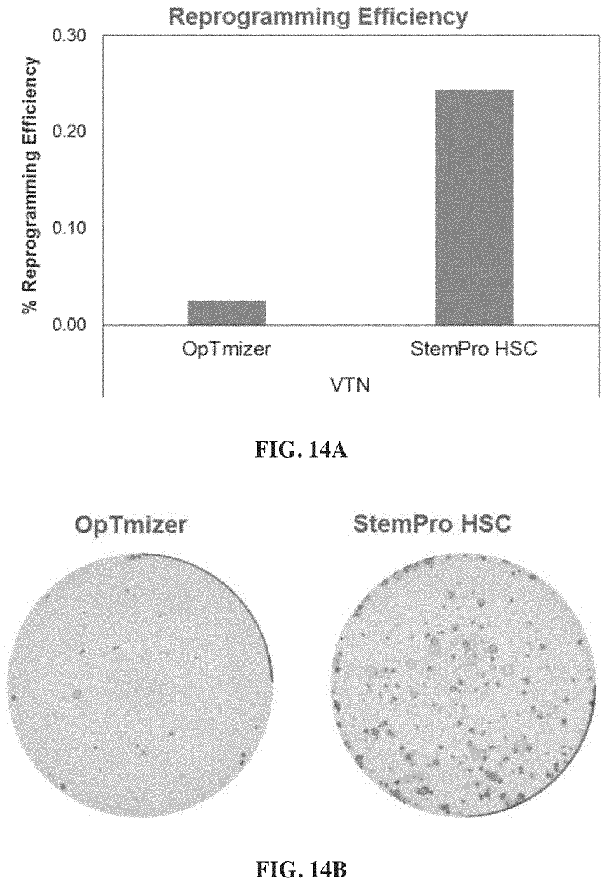

Due to the potential of iPSCs in a therapeutic context, a xeno-free reprograming workflow is desirable. For example, xeno-free workflows are desirable from a regulatory standpoint. The following experiment demonstrates that HSC's can be reprogrammed to iPSCs using the xeno-free HSC expansion media as described herein. Single donor, cord-blood derived, CD34+ cells from 2 individuals were thawed and cultured in either OPTMIZER™ cell media (Thermo Fisher Scientific) containing SCF, IL-3, and GM-CSF; or STEMPRO™ HSC Expansion media as described herein containing SCF, Flt3L, TPO, IL-3, and IL-6. During each day of culture, half the media was removed and replaced with fresh media containing cytokines. Cells were transduced with CTS™ CytoTune™ 2.1-iPS Sendai Reprogramming Kit (Thermo Fisher Scientific) according to the manufacturer's protocol, at an MOI of 5-5-3 (KOS-LMyc-Klf4) in the presence of polybrene. Three days later, cells were plated onto recombinant human vitronectin (rhVTN-N) coated plates in HSC Expansion media as described herein containing no cytokines. Seven days after transduction, half the media was removed and replaced with ESSENTIAL 8™ Medium (Thermo Fisher Scientific). The following day, and every day thereafter, cells were fed with ESSENTIAL 8™ Medium. Sixteen days after transduction, reprogramming efficiency was determined by staining cells for Alkaline Phosphatase using the Vector Red Alkaline Phosphatase Substrate Kit. The number of AP positive colonies was counted, and reprogramming efficiency was determined relative to the number of cells plated on Day 3 after transduction. Shown is the mean reprogramming efficiency (FIGS. 14A, 14B).

### Example 6: StemPro HSC Expansion Medium (Prototype) Enables Reprogramming of PBMC into iPSC with CTS CytoTune 2.1 and CytoTune 2.0

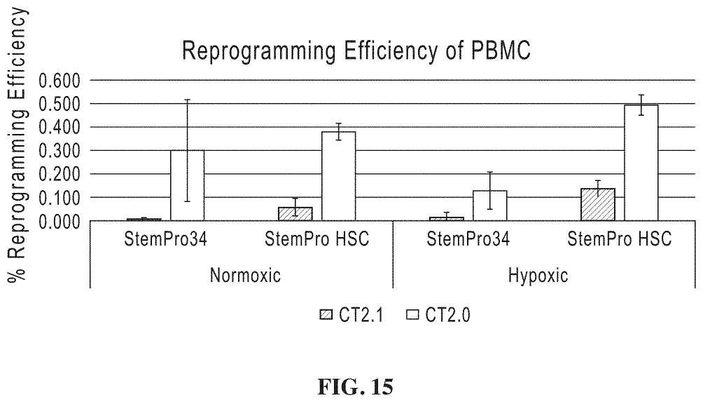

The following experiment demonstrates that HSC's can be reprogrammed to iPSCs using the xeno-free HSC expansion media as described herein. Single donor, PBMCs CD34+ cells from three single donors were obtained, thawed and cultured in either STEMPRO™ 34 cell media (Thermo Fisher Scientific) containing SCF, IL-3, and GM-CSF; or HSC Expansion media as described herein containing SCF, Flt3L, TPO, IL-3, and IL-6. During each day of culture, half the media was removed and replaced with fresh media containing cytokines. Cells were transduced with CTS™ CytoTune™ 2.1-iPS Sendai Reprogramming Kit (Thermo Fisher Scientific) according to the manufacturer's protocol, at an MOI of 5-5-3 (KOS-LMyc-Klf4) in the presence of polybrene. Following transduction, the cells were split into two cultures; one culture was grown in normoxic conditions, the other culture was grown in hypoxic conditions, in the STEMPRO™ 34 media or HSC Expansion media with growth factors as described above. Three days later, cells were plated onto recombinant human vitronectin (rhVTN-N) coated plates in STEMPRO™ 34 media or HSC Expansion Media as described herein containing no cytokines. Seven days after transduction, half the media was removed and replaced with ESSENTIAL 8™ Medium (Thermo Fisher Scientific). The following day, and every day thereafter, cells were fed with ESSENTIAL 8™ Medium. Sixteen days after transduction, reprogramming efficiency was determined by staining cells for Alkaline Phosphatase using the Vector Red Alkaline Phosphatase Substrate Kit. The number of AP positive colonies was counted, and reprogramming efficiency was determined relative to the number of cells plated on Day 3 after transduction. Shown is the mean efficiency for the three donors (FIG. 15).

### Example 7: Engraftment of CD34+ Cells Expanded in HSC Expansion Media

Human CD34+ Cells are obtained and expanded in HSC expansion media described herein for 7 days. Various concentrations of expanded cells, unexpanded cells (control) are transplanted into lethally irradiated immunodeficient mice. At different timepoints (2 months, 6 months), mice are euthanized and their bone marrow cells and spleen cells are harvested. Bone marrow and spleen cells are stained with antibodies against human CD34, CD33, CD45, Lineage, CD3 and CD19 cell surface markers and analyzed using flow cytometer. Mice transplanted with CD34+ cells expanded in HSC expansion medium as described herein have an increased number of engrafted cells, compared to mice transplanted with unexpanded cells, demonstrating improved engraftment capacity. Engraftment is observed at both 2 months (short term engraftment) and at 6 months (long term engraftment).

To demonstrate that the transplanted cells have self-renewing capacity, bone marrow cells of mice euthanized at 6 months as described above are transplanted into lethally irradiated immunodeficient mice (secondary transplant). At two months, the mice are euthanized and their bone marrow cells and spleen cells are harvested. Bone marrow and spleen cells are stained with antibodies against human CD34, CD33, CD45, Lineage, CD3 and CD19 cell surface markers and analyzed using flow cytometer. Presence of transplanted cells in bone marrow and spleen is observed, indicating self-renewal capacity of expanded cells.

### Example 8: Additional HDAC Inhibitors and HAT Inhibitors

The ability of additional HDAC inhibitors and/or HAT inhibitors to increase expansion of HSCs is evaluated. One or more HDAC inhibitors and/or HAT inhibitors are tested, alone or in combination. It is expected that additional HDAC inhibitors and HAT inhibitors will have a similar effect on the expansion of HSCs.

### Example 9: Effect of HSC Medium on Primary Cell Expansion

The ability of HSC medium to promote expansion of primary cells derived from a human donor is evaluated. Different types of primary cells (e.g., macrophages, T cells) are expanded in HSC medium as described herein. It is expected that the HSC medium will increase expansion of some additional primary cell types, for example T cells.

### Example 10: Treatment of Patients with HSCs Expanded in HSC Medium

The ability of HSCs expanded as described herein to treat a patient having a disease treatable by HSCs is determined. HSCs are derived from an HSC source that is allogenic for the patient to be treated. HSCs are expanded ex vivo in HSC Medium. Expanded HSCs are administered to the patient in an amount effective to promote engraftment of the cells and treatment of the condition.

1. A growth medium for culture of hematopoietic stem cells (HSCs), comprising a basal medium and a supplement, the medium and/or supplement comprising a histone acetyltransferase (HAT) inhibitor, a histone deacetylase (HDAC) inhibitor and two or more of a lipid, an amino acid or amino acid derivative, an antioxidative agent, and an inorganic salt.

2. The growth medium of claim 1, wherein the basal medium is selected from OPTMIZER™ CTS™ T-Cell Expansion serum-free medium, Dulbecco's Modified Eagle Media (DMEM), Iscove's Modified Dulbecco's Medium (IMDM), DMEM/F12, Advanced DMEM/F12, and KNOCKOUT™ DMEM/F12.

3. The growth medium of claim 1, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin.

4. The growth medium of claim 1, wherein the HAT inhibitor is present at a concentration of between 0.001 grams/liter (g/L) and 1 g/L.

5. The growth medium of claim 4, wherein the HAT inhibitor is present at a concentration of between 0.001 grams/liter (g/L) and 0.005 g/L.

6. The growth medium of claim 1, wherein the HDAC inhibitor is selected from apicidin, belinostat, CI-994, CRA-024781, panobinostat, sodium butyrate, sodium phenylbutyrate, suberoylanilide hydroxamic acid (vorinostat), trichostatin A, sodium valproate (valproic acid), givinostat, MS-275, MGCD0103, and Scriptaid.

7. (canceled)

8. The growth medium of claim 1, wherein the HDAC inhibitor is present at a concentration between 0.01 g/L and 0.1 g/L.

9. The growth medium of claim 1, wherein the HDAC inhibitor and HAT inhibitor are at a weight ratio of 1:1 to 1:30 HDAC inhibitor:HAT inhibitor.

10-11. (canceled)

12. The growth medium of claim 1, further comprising albumin, insulin, transferrin, interleukin 3 (IL-3), interleukin 6 (IL-6), stem cell factor, Fms-related tyrosine kinase 3 ligand, thrombopoietin, granulocyte colony-stimulating factor, granulocyte-macrophage colony-stimulating factor, and combinations thereof.

13. (canceled)

14. The growth medium of claim 1, wherein the medium does not comprise an ingredient derived from an animal or is a xeno-free medium.

15-33. (canceled)

34. A growth medium supplement for the culture of hematopoietic stem cells (HSCs), comprising a histone acetyltransferase (HAT) inhibitor and a histone deacetylase (HDAC) inhibitor.

35. The growth medium supplement of claim 34 further comprising a lipid, an amino acid or amino acid derivative, an antioxidative agent, and an inorganic salt.

36. The growth medium supplement of claim 34, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin.

37-38. (canceled)

39. The growth medium supplement of claim 34, wherein the HDAC inhibitor is selected from apicidin, belinostat, CI-994, CRA-024781, panobinostat, sodium butyrate, sodium phenylbutyrate, suberoylanilide hydroxamic acid (vorinostat), trichostatin A, sodium valproate (valproic acid), givinostat, MS-275, MGCD0103, and Scriptaid.

40-41. (canceled)

42. The growth medium supplement of claim 34, comprising a HDAC inhibitor and a HAT inhibitor at a weight ratio of 1:1 to 1:30 HDAC inhibitor:HAT inhibitor.

43-46. (canceled)

47. A method of expanding stem cells, the method comprising growing stem cells in a growth medium comprising a basal medium, a histone acetyltransferase (HAT) inhibitor, a histone deacetylase (HDAC) inhibitor, and two or more of a lipid, an amino acid or amino acid derivative, an antioxidative agent, and an inorganic salt.

48. The method of claim 47, wherein the stem cells are hematopoietic stem cells (HSCs).

49-56. (canceled)

57. The method of claim 47, wherein the HAT inhibitor is selected from 2,6-Bis[(3-bromo-4-hydroxyphenyl)methylene]cyclohexanone, MG149, C646, CPTH2, curcumin, A-485, anacardic acid, MB-3, and chalcones such as garcinol, isogarcinol, xanthohumol, isoxanthohumol, 2-hydroxycalchone, 4-hydroxycalchone, yakuchinone A, and isoliquiritigenin.

58-59. (canceled)

60. The method of claim 47, wherein the HDAC inhibitor is selected from apicidin, belinostat, CI-994, CRA-024781, panobinostat, sodium butyrate, sodium phenylbutyrate, suberoylanilide hydroxamic acid (vorinostat), trichostatin A, sodium valproate (valproic acid), givinostat, MS-275, MGCD0103, and Scriptaid.

61-62. (canceled)

63. The method of claim 47, wherein the HDAC inhibitor and HAT inhibitor are present in the growth medium at a weight ratio of 1:1 to 1:30 HDAC inhibitor:HAT inhibitor.

64-65. (canceled)

66. The method of claim 47, the growth medium further comprising albumin, insulin, transferrin, interleukin 3 (IL-3), interleukin 6 (IL-6), stem cell factor, Fms-related tyrosine kinase 3 ligand, thrombopoietin, granulocyte colony-stimulating factor, granulocyte-macrophage colony-stimulating factor, and combinations thereof.

67. (canceled)

68. A method of expanding stem cells, the method comprising (i) adding the growth medium supplement of claim 34 to a basal medium to form a growth medium comprising the HAT inhibitor and the HDAC inhibitor, and (ii) growing stem cells in the growth medium, thereby expanding the stem cells.

69-98. (canceled)
